(19) United States
(12) Patent Application Publication
(10) Pub. No.: US 2002/0142417 A1
(43) Pub. Date: Oct. 3, 2002

(54) Antioxidant protein 2, gene and methods of use therefor

(76) Inventors: Beverly Paigen, Bar Harbor, ME (US); David R. Beier, Chestnut Hill, MA (US)

Correspondence Address: Farrell & Associates, P.C., P.O. Box 999, York Harbor, ME, 03911, US

(21) Appl. No.: 09/819,505

(22) Filed: Mar. 28, 2001

Related U.S. Application Data
(62) Division of application No. 09/053,088, filed on Apr. 1, 1998.
(60) Provisional application No. 60/040,898, filed on Apr. 2, 1997.

(51) Int. Cl. C12N 9/00; A61K 39/00; C12P 21/02; C12N 5/06

(52) U.S. Cl. 435/183; 424/185.1; 435/69.1; 435/320.1; 435/325

(57) ABSTRACT

The present invention involves the identification of a novel gene and protein, now designated as antioxidant protein 2 (Aop2 and AOP2, respectively). Studies indicate that Ltw4 and Aop2 are a single gene. In addition, Aop2 also appears to be the gene responsible for the Athl trait in mice—a predisposition to atherosclerotic disease. The human homolog for this gene also has been identified. This discovery makes possible a variety of uses for AOP2 and the corresponding gene, for example, development of reagents (antibodies, expression vectors, cell lines, congenic and transience mice) that may be used in the diagnosis and treatment of atherosclerosis and related disease states.

The government may own rights to this application or any patent issued thereto under the following grants: HL-32087 and DK-45639 from the National Institutes of Health.

### BACKGROUND OF THE INVENTION

I. Field of the Invention

The present invention relates to the fields of medicine, molecular biology and biochemistry. More particular, the invention relates to the cloning, analysis and use of genes related to atherosclerotic disease, a clinically significant condition that may lead to heart attacks and strokes. Specifically, the invention relates, in one embodiment, to the novel gene and gene product, Aop2 and AOP2, respectively, and to the novel role of AOP2 as an antioxidant in the protection against atherosclerotic disease.

II. Related Art

Atherosclerosis is a major contributing cause of heart disease, the leading cause of death in the United States. It is characterized by the formation of elevated intimal fibrofatty plaques called atheromas, which narrow arterial lumens, damage the underlying media and are prone to undergo superimposed complications including calcification, ulceration with overlying thrombosis and intraplaque hemorrhage. The centers of these plaques often contain a lipid-rich debris containing notably cholesterol and cholesterol esters.

Atherosclerosis has achieved considerable medical importance because of its predilection for the coronary arteries and the arterial supply to the brain and the heart.

Atheromatous involvement of the coronary arteries underlies the dominant form of heart disease, i.e., coronary heart disease, also known as ischemic heart disease, the most important form of which is myocardial infarction. Ischemic heart disease represents about 90% of all forms of heart disease, and myocardial infarction is the commonest cause of death in atherosclerosis-prone populations.

Atherosclerosis is virtually ubiquitous among the populations of North America, Europe and the Soviet Union. In contrast, it is much less prevalent in Central and South America, Africa, Asia and the Orient. A comparison of Finland, which has the highest mortality rate from ischemic heart disease, with Japan shows a differential of over ten-fold. The U.S. ranks in the top ten in mortality from ischemic heart disease. Though it is clear that genetic predisposition exists, the primary focus of diagnosis and treatment relates to environmental influences, particularly smoking, sedentary lifestyles, obesity, hypertension, diet and plasma cholesterol levels.

It is well known that elevated levels of cholesterol in the plasma carry an increased risk of heart disease. However, this cholesterol is carried in both low density lipoproteins (LDL-C) and high density lipoproteins (HDL-C). It is the levels of LDL-C that carry the increased risk to heart disease; elevated levels of HDL-C actually protect from atherosclerosis and heart disease. To understand the mechanism of how elevated LDL-C leads to the formation of atherosclerosis in the arteries, one must turn to the earliest events in the formation of the fatty streak, which is the precursor of the atherosclerotic plaque. The first step in the formation of a fatty streak is the accumulation of foam cells underneath the endothelial layer of the artery. Foam cells are monocyte-derived macrophages filled with fat, and these cells are the major cell type in fatty streaks. The key step that leads to the formation of foam cells is the oxidation of LDL-C (reviewed by Steinberg et al., 1989; Netto et al., 1996; Witzum & Steinberg, 1991). Macrophages take up primarily oxidized LDL, so it is oxidized LDL that leads to the formation of foam cells and fatty streaks. Dietary antioxidants, such as vitamin E or natural antioxidants in the body could protect LDL from oxidation. HDL-C also protects LDL-C from oxidation (reviewed by Banka, 1996), but in the process HDL itself becomes oxidized. When oxidized, HDL carries most of the lipid peroxidation products in plasma (Bowry et al., 1992; Hahn et al., 1994) and its apoproteins are cross-linked by oxidative damage (Marcel et al., 1989). This damaged and oxidized HDL is cleared from the plasma more quickly than native HDL (Guertin et al., 1994; Senault et al, 1990). The consequences of HDL protection of LDL from oxidation results in relatively high plasma HDL if very little oxidation of LDL is occuring, but if more oxidation is occurring, then HDL becomes oxidized, cleared from the plasma more rapidly, and plasma levels of HDL decrease.

Atherosclerosis is not unique to human populations. Athl is a previously described gene locus that affects the concentration of high density lipoprotein cholesterol and susceptibility to atherosclerosis among inbred strains of mice. The atherosclerosis-susceptible allele is carried by strain C57BL/6J and the resistant alleles are carried by strains C3H/HeJ and BALB/cJ. The susceptible phenotype of Athl is characterized by relatively low concentrations (35-45 mg/dl) of high density lipoprotein-cholesterol (HDL) in the plasma of female mice fed a high fat and high cholesterol diet and the development of fatty streak lesions at many places along the aorta (Paigen, 1985). These fatty streaks eventually develop into mature atherosclerotic plaques (Paigen et al., 1990). The resistant phenotype of Athl, found in strains BALB/c and C3H, is characterized by higher concentrations (60-90 mg/dl) of plasma HDL and the absence of fatty streak lesions in the aorta when mice are fed the atherogenic diet.

The Athl phenotype is caused by a single gene difference between strains C57BL/6 and C3H and also between strains C57BL/6 and BALB/c. This conclusion was initially based on examination of recombinant inbred (RI) strains BXH and CXB (Paigen, 1987a) and confirmed by backcrosses between C57BL/6 and the two resistant strains (Paigen, 1987b, 1987c). Athl mapped to the distal end of mouse chromosome 1 at a distance of 6 cM from the gene for apolipoprotein AII (Apoa2). Athl was clearly separate from Apoa2 as demonstrated by analysis of a congenic strain, B6.C-H25^{c}/By, which carries the apoAII gene from BALB/c but the Athl susceptible gene from B6 (Paigen 1987a). Athl maps to the region homologous to chromosome 1q24-25 in humans.

Despite this useful information, there is very little direct knowledge regarding the role of this gene in atherosclerotic disease. Not only is the identity of the gene unknown, its function remains purely speculative. As a result, there is a considerable need in the field for evidence that will establish a fictional link between Athl and heart disease, as well as information on the structural attributes of the gene product of Athl.

### SUMMARY OF THE INVENTION

Therefore, it is a goal of the present invention to provide additional information on the role of the Athl gene in atherosclerotic disease. It is another goal of the present invention to provide compositions and methods for use in diagnosing and treating Athl related disease states. It also is a goal of the present invention to develop reagents that can stimulate or inhibit functions related to Athl so to address pathologic states associated therewith.

In satisfying these objectives, there is provided an isolated polypeptide designated AOP2. The polypeptide may be from any species, preferably mammalian, and more preferably human or murine. In one specific embodiment, the polypeptide has the sequence set forth in SEQ ID NO:2. In another specific embodiment, the polypeptide has the sequence set forth in SEQ ID NO:4.

In another embodiment, there is provided an antigen composition comprising an Aop2 polypeptide or a fragment thereof and a pharmaceutically acceptable buffer or diluent. Again, the polypeptide may be human or murine, and more specifically may be derived from the sequence of SEQ ID NO:2 or SEQ ID NO:4 respectively.

In still another embodiment, there is provided a nucleic acid encoding an AOP2 polypeptide. The nucleic acid may encode a human polypeptide or a murine polypeptide, among other mammalian and non-mammalian polypeptides. The nucleic acid may encode a polypeptide of SEQ ID NO:2 or SEQ ID NO:4 and, more specifically, the nucleic acid may have a sequence as set forth in SEQ ID NO:1 or SEQ ID NO:3.

In still yet another embodiment, there is provided an oligonucleotide comprising at least about 10 consecutive bases of the nucleic acid sequence set forth in SEQ ID NO:1 or SEQ ID NO:3. The oligonucleotide may be longer, for example, at least about 15, 20, 25, 30, 35, 40, 45 or 50 consecutive bases of the nucleic acid set forth in SEQ ID NO:1 or SEQ ID NO:3.

In still yet a further embodiment, there is provided a method for diagnosing a predisposition to atherosclerotic lesions in a subject comprising (i) obtaining a sample from said subject; and (ii) evaluating said sample for the presence of an AOP2 polypeptide. The sample may be selected from the group consisting of heart, artery, vein, skin, muscle, facia, brain, prostate, breast, endometrium, lung, pancreas, small intestine, blood cells, liver, testes, ovaries, colon, skin, stomach, esophagus, spleen, lymph node, bone marrow or kidney, lymph fluid, ascites, serous fluid, pleural effusion, sputum, cerebrospinal fluid, lacrimal fluid, stool and urine. The subject may be a human.

The evaluating may comprise determining the antioxidant activity of an AOP2 polypeptide of said sample, determining the level of an AOP2 polypeptide in cells of said sample or determining the sequence of a nucleic acid from said sample that encodes an AOP2 polypeptide. The determining may comprise quantitative PCR or contacting said sample with an antibody that binds immunologically to an AOP2 polypeptide.

In still yet a further embodiment, there is provided a method for screening a compound for AOP2 stimulatory activity comprising (i) providing an AOP2 polypeptide having antioxidant activity; (ii) contacting said AOP2 polypeptide with a candidate stimulator; and (iii) determining the antioxidant activity of said AOP2 polypeptide in the presence and absence of said candidate stimulator.

In yet another embodiment, there is provided a method for screening a compound for antioxidant stimulatory activity comprising (i) providing a cell comprising an nucleic acid encoding an active AOP2 polypeptide; (ii) contacting said cell with a candidate stimulator; and (iii) determining the antioxidant activity in said cell in the presence and absence of said candidate stimulator. The cell may be located in a non-human animal.

In still yet another embodiment, there is provided a method for screening a compound for anti-atherosclerotic activity comprising (i) providing a lipid; (ii) contacting said lipid with a candidate antioxidant; and (iii) determining the oxidation state of said lipid.

In still yet other embodiments, there are provided a monoclonal antibody that binds immunologically to an AOP2 polypeptide and a polyclonal antisera, antibodies of which bind immunologically to an AOP2 polypeptide.

Yet further embodiments include an expression vector comprising a nucleic acid encoding an AOP2 polypeptide, said nucleic acid positioned in operable relation to a promoter and a recombinant host cell comprising a nucleic acid encoding an AOP2 polypeptide, said nucleic acid positioned in operable relation to a promoter.

In still a further embodiment, there is provided a method for increasing AOP2 function in a cell comprising (i) providing a nucleic acid encoding an AOP2 polypeptide having antioxidant activity, said nucleic acid positioned in operable relation to a promoter; and (ii) contacting said nucleic acid with said cell under conditions permitting the uptake of said nucleic acid. The AOP2 polypeptide may be a human polypeptide, for example, as set forth in SEQ ID NO:2. The nucleic acid further may comprise an expression vector, and the expression vector may be encapsulated in a liposome. The expression vector may be a viral vector, such as an adenoviral vector, a retroviral vector, a vaccinia viral vector, an adeno-associated viral vector or a herpesviral vector. The cell may be located in a human subject, in an experimental animal. The nucleic acid may be administered intravenously. The promoter may be CMV, RSV or E1A.

In another embodiment, there is provided a method of reducing atherosclerotic lesions in a subject comprising administering to said subject a compound with antioxidant composition. It is noted that an antioxidant may function to protect lipid directly or indeed break down free radicals that could oxidize a lipid composition, thereby protecting the lipid indirectly.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1: LTW4 protein isoforms. Corresponding regions of silver-stained two-dimensional electrophoresis gels of liver homogenates from C57BL/6 (top panel) and DBA/2 (bottom panel) are shown. The C57BL/6 isoform of LTW4 has a MW of 26 kD and a pI of 5.9. The DBA/2 isoform of LTW4 has a MW of 26 kD and a pI of 5.6.

FIG. 2: Consensus sequence for Aop2. Nucleotide 389 (shown in bold) is adenine in DBA/2J, C3H and BALB/c and cytosine in C57BL/6J. The codon containing this polymorphic nucleotide corresponds to aspartic acid in DBA/2J and to alanine in C57BL/6J. The N-terminal amino acid sequence obtained by microsequence analysis is underlined.

FIGS. 3A & 3B: SSCP mapping analysis of Aop2. (FIG. 3A) Analysis of genomic DNA from parental strain controls and 22 progeny from the BSS interspecific cross using an SSCP derived from 3′ UTR of Aop2. Since this is a (C57BL/6J X M. spretus) F1 X M. spretus backcross, all progeny carry at least one M. spretus allele. Heterozygotes also carrying a C57BL/6J allele are marked. B: C57BL/6J control; S: M spretus control; H: C57BL/6J -M. spretus heterozygote. (FIG. 3B) The position of Aop2 in the BSS cross is shown on the left, and is compared to the position of Ltw4 on the chromosome 1 consensus map¹⁰ on the right. Interlocus distances are shown in cM.Apoa2:apolipoprotein AII.

FIG. 4: Amino acid homologies of murine anti-oxidant proteins. Four members of the murine antioxidant protein family were analyzed using the Blocks program (http://www.blocks.fhcrc.org¹⁸). Three domains of conserved amino acids were identified and are shown. Amino acid identities are shown in bold. The two cysteines that are conserved in most members of the TSA family are marked with an asterisk; the second cysteine is not present in AOP2 or its human homologue.

FIG. 5: High resolution map of mouse chromosome 1. Polymorphic markers illustrated.

FIG. 6: (A) Genomic structure of the mouse Aop2 gene. Exons are represented by boxes; light portions indicated untranslated regions and dark portions indicate the coding region. The relative proportion of exon and intron sizes are shown. Five exons and four introns span approximately 10.7 kb. (B) Sizes of exons and introns and splice junction sequences.

FIG. 7: Nucleotide sequence of the 5′-flanking region of the mouse Aop2 gene. Nucleotide position +1 corresponds to the 5′-terminus of the mouse mGPx cDNA (Munz et al., J. Biochem. 326: 579-585 (1997)), and negative numbers refer to 5′ flanking sequences. The translation start codon ATG is underlined, and putative transcription factor binding sites are boxed.

FIG. 8: (A) Alignment of the Aop2 nucleotide sequence with that of the Aop2-rs1 and Aop2-rs2. Reported sequences span the corresponding coding region of Aop2. The arrow indicates the start of translation. Dashes represent nucleotide identities with Aop2; dots represent gaps in the sequence. (B) Alignment of the Aop2 protein with the predicted amino acid sequences encoded by Aop2-rs1 and Aop2-rs2. Dashes represent amino acid identities with Aop2.

### DETAILED DESCRIPTION OF THE PREFERRED EMBODIMENTS

### I. The Present Invention

The present invention relates to the identification of a novel gene, designated Aop2, which maps to the previously described atherosclerosis susceptibility locus, Athl. The Aop2 gene product, now designated AOP2 is an antioxidant and belongs to a highly related family of antioxidant proteins. The inventors have identified two different alleles of Athl responsible for a difference in atherosclerosis susceptibility between mouse strains. Sequencing of Aop2 cDNA from susceptible and resistant mouse strains identified a single base pair difference in the sequences of the two alleles. The inventors further have determined the structure of the Aop2 gene. The Athl resistant allele, carried by inbred mouse strains C3H and BALB/c, must code for an AOP2 protein that functions well as an antioxidant while the Athl susceptible allele, carried by strain C57BL/6, must code for an AOP2 protein that functions less well as an antioxidant, permitting more LDL to be oxidized, more foam cells to form, more fatty streaks to raise, and more HDL-C to be damaged by oxidation products, thus resulting in more rapid clearance of HDL and lower HDL levels in plasma. In other embodiments, it has been demonstrated that phenotypic differences may also result from differences in Aop2 expression and not just protein structure. More specifically, data discussed in new Example 7 reveals differences in Aop2 mRNA levels. Example 8 discloses genomic sequence information including potentially important regulatory elements in the promoter region.

Using a high resolution mapping cross of over 1000 mice, the inventors were able to localize the atherosclerosis susceptibility locus, Athl, to narrow a genetic region on mouse chromosome 1, with no recombinants evident with the locus D1Mit424. The cross was between C57BL/6, a strain that is susceptible to atherosclerosis, and a congenic strain constructed by several generations of backcrossing the atherosclerosis resistant strain Spretus to C57BL/6. The congenic strain carries mostly C57BL/6 genes except for a small region of Spretus genes on chromosome 1, extending at least from the markers D1Mit14 to D1Mit15. Heterozygous mice from a mating between C57BL/6 and the congenic strain, named C57BL/6.Sp-Athl^{r}, were mated to C57BL/6. The resulting backcross progeny were phenotyped for the size of their fatty streak lesions in the aorta and genotype for the loci in the relevant region of mouse chromosome 1. Thus, Athl was narrowed to a very small region.

One gene that maps to this region is Ltw4, identified previously by variant polypeptides using two-dimensional gel electrophoresis (Elliott, 1979a; Elliott, 1979b; Elliott et al., 1980). This protein is expressed in the liver and has a molecular weight of 20-30 kD. The inventors used analytical and preparative two-dimensional gel electrophoresis and identified variants of a 26 kD polypeptide which differed in their isoelectric point between strains C57BL/6 and DBA/2 in a pattern similar to that originally described for LTW (Iakoubova et al., 1997). N-terminal amino acid sequence was obtained by microsequencing, and overlapping expressed sequence tags (ESTs) corresponding to a full-length coding sequence were identified. Confirmation of identity was made by mapping the gene using single-strand conformational polymorphism (SSCP); the map position corresponded to that previously described for Ltw4. This gene then was mapped in the same mice used for the high resolution mapping of Athl; no recombinants were found between this gene and Athl, strongly suggesting that Athl and Ltw4 are the same.

The cDNA corresponding to Ltw4 was sequenced and found to have homology to a class of proteins characterized as thiol-specific antioxidants that are protective against damage caused by oxidative stress (Chae et al., 1994). The murine MER5 gene also is a member of this gene family (Yamamoto et al., 1989) and recently has been renamed antioxidant protein 1, or Aop1, based on its functional characterization (Tsuji et al., 1995). Hence, the inventors propose that the Ltw4 gene be designated as antioxidant protein 2, or Aop2, and the corresponding protein be designated as AOP2.

The cDNA for Aop2 was sequenced from atherosclerotic resistant and susceptible strains and a subset found to differ at a single base, nucleotide 389, that corresponds to an amino acid change in residue 124. This residue is the amino acid alanine in C57BL/6 mice, which are susceptible to atherosclerosis, and aspartic acid in C3H mice, which are resistant to atherosclerosis. This amino acid change results in a change in isoelectric point that corresponds to the differences observed for LTW4.

Thus, the inventors propose that the difference in atherosclerosis susceptibility caused by Athl is determined by an antioxidant protein, AOP2, which differs between resistant and susceptible strains. This proposal is based on the (i) concordance of Aop2 with Athl in a high resolution mapping cross, (ii) the isolation and sequencing of cDNA that differs between Athl susceptible and Athl resistant strains, (iii) the observation that the respective protein products differ by an amino acid between the susceptible and resistant strains, and (iv) the biological plausability of an antioxidant having an important role in atherosclerosis, given that oxidation of plasma lipoproteins is of major importance in the atherosclerotic process.

The hypothesis that AOP2 differs in function between the resistant and susceptible strains further finds credibilty in the fact that oxidized LDL causes more foam cells to form, and hence more fatty streaks to develop. HDL protects LDL against oxidation, and when HDL becomes damaged by oxidation products, it is cleared from the plasma more readily. Significantly, the major differences between the Athl resistant and susceptible strains are in the size of the fatty lesions and the levels of plasma HDL.

With the identification of the Aop2 gene as a contributing factor to atherosclerosis, a number of different uses become possible. For example, by screening for abnormalities in this gene, it now is possible to ascertain, at the genetic or protein level, a predisposition to atherosclerotic disease. In addition, the association of this gene with antioxidant activity suggests the use of Athl mice strains to screen for compounds that modulate antioxidant activity in vivo.

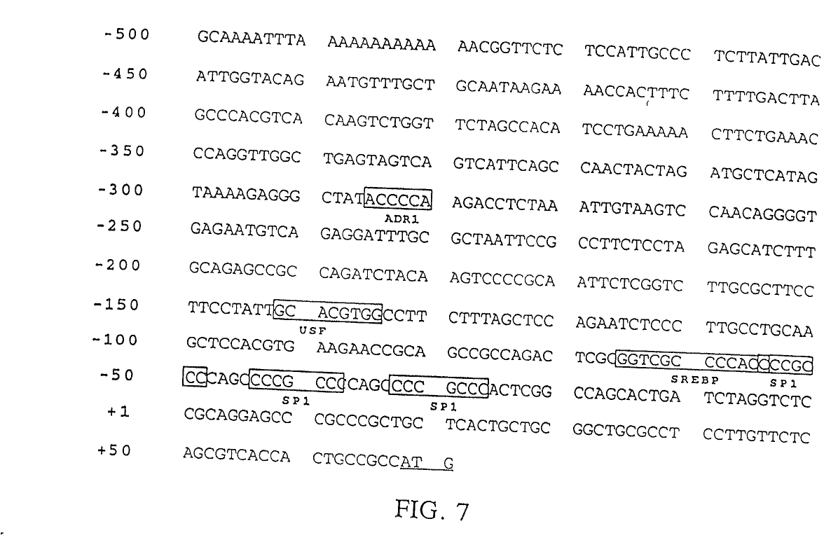

It also is possible to employ both protein and DNA compositions in the treatment of oxidative damage, atherosclerosis and heart disease. With respect to the latter application, the identification of important, native, genomic regulatory elements, particularly those associated with increased expression, is of paramount importance. The sequence information presented in Example 8 enables the identification of such critical regulatory sequences. More specifically, FIG. 7, described in Example 8, describes several consensus recognition sequences for known transcription factors are found in the putative proximal promoter. These include potential binding sites for USF (upstream stimulatory factor) and SREBP (sterol response element binding protein). Both of these DNA biding proteins have been demonstrated to be important in the regulation of gene expression. Although the particular promoter sequence described is not associated with particularly high Aop2 transcription levels, given the identification of the consensus regulatory sequences, one of skill in the art could isolate promoter sequences from alleles characterized by increased levels of mRNA production and compare such sequences with that disclosed in FIG. 7. Such analysis is likely to reveal the identity of important regulatory sequences responsible for the differences in Aop2 expression documented herein.

The identification of such regulatory sequences can be exploited therapeutically in a gene therapy protocol. AOP2 activity appears to be localized to the cytosol. A therapeutic approach for the treatment of an Athl susceptible individual is to introduce a mammalian expression vector carrying an Aop2 allele conferring Athl resistance to the relevant cells of the individual. With respect to atherosclerosis, the relevant target cells would be the cells of the artery wall (the site of low density lipoprotein (LDL) oxidation) Conventional mammalian expression vectors are discussed in greater detail below. Previous work has shown that the delivery of genes by such conventional mammalian vectors can result in expression in the cells of the artery wall. In this context, it is important to note that Aop2 is widely expressed in at least 23 distinct tissues. In light of this somewhat ubiquitous expression pattern, deleterious effects associated with expression of Aop2 in cells other that the target cells would be highly unlikely.

### II. AOP2 Polypeptides and Fragments Thereof

Thus, according to one aspect of the present invention, the present inventors provide the primary sequence for the polypeptide AOP2. This molecule will prove useful in a variety of different contexts. For example, AOP2 may be used as an antigen to raise antibodies that can, in turn, be used to study and diagnosis disease states relating to AOP2. AOP2 also can be used as part of screening assays to examine reagents for their ability to affect the function of AOP2 in vitro or in vivo. Finally, AOP2 may be used to prevent oxidative damage in vivo, for example, during heart surgery where blood is oxygenated outside the body.

In addition to the entire AOP2 molecule, the present invention also relates to fragments of the polypeptide that may or may not retain the antioxidant (or other) activity. Fragments including the N-terminus of the molecule may be generated by genetic engineering of translation stop sites within the coding region (discussed below). Alternatively, treatment of the AOP2 molecule with proteolytic enzymes, known as protease, can produces a variety of N-terminal, C-terminal and internal fragments. Examples of fragments may include contiguous residues of the Athl sequences given in SEQ ID NO:2 or SEQ ID NO:4, of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 200, 300, 400 or more amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

### A. Structural Features of the Polypeptide

The gene for Aop2 encodes a 224 amino acid polypeptide. The predicted molecular weight of this molecule is 26 kD. Thus, at a minimum, this molecule may be used as a standard in assays where molecule weight is being examined.

### B. Functional Aspects

When the present application refers to the function of Aop2 or “wild-type” activity, it is meant that the molecule in question has the ability to prevent oxidation of various biological molecules such as lipids, proteins and especially low and high density lipoproteins. Specifically, AOP2 is a member of a family of proteins that can protect enzymes from damage by thiol-dependent, metal catalyzed oxidation. Determination of which polypeptides possess this activity may be determined using assays familiar to those of skill in the art. For example, transfer of genes encoding Aop2, or variants thereof, into cells that do not have a functional Aop2 product, and hence exhibit excess oxidation, will identify those molecules having AOP2 function.

For example, cell extracts may be assayed for protection of enzymes from metal-catalyzed oxidation. There also is at least one yeast strain deficient in AOP2-like function which may be used in complementation studies with Aop2 genes. In a particular example, the protein could be measured using an immunoassay with an antibody specific to that protein. The activity of this protein also could be measured by its ability to protect LDL from oxidation. Such assays are based on the quantity of oxidized LDL; if the antioxidant is functioning well, the quantity of oxidized LDL will be low and conversely if the antioxidant has poor function, oxidized LDL will be in high quantity. Oxidized LDL can be estimated directly by measuring the amount of thiobarbituric acid reactive substances (TBARS), with absorbance at 532 nm, that were generated (Ohkawa et al., 1979). This assay is used extensively in atherosclerosis research (Parthasarathy et al., 1990). Another method of direct estimation of oxidized LDL is to measure the products of lipid peroxidation, which are conjugated dienes and trienes, as described (Klimov et al., 1993). An indirect method of measuring oxidized LDL is uptake by macrophages. Since macrophages take up oxidized LDL but not native LDL, the ability of macrophages to degrade¹²⁵I-labeled LDL is used (Parthasarathy et al., 1990). Finally, members of this class of antioxidants can be assayed by their ability to protect glutamine synthetase from oxidative inactivation by thiol-dependent metal catalyzed oxidations systems (Netto et al., 1996). This involves removal of hydrogen peroxide from the oxidation system.

### C. Variants of AOP2

Amino acid sequence variants of the polypeptide can be substitutional, insertional or deletion variants. Deletion variants lack one or more residues of the native protein which are not essential for function or immunogenic activity. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (-0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0 ±1); glutamate (+3.0 ±1); serine (+0.3); asparagine (+0.2) glutamine (+0.2); glycine (0); threonine (−0.4); proline (−0.5 ±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within +2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of AOP2, but with altered and even improved characteristics.

### D. Domain Switching

As described in the examples, the present inventors have identified putative murine and human homologues of the Aop2 gene. In addition, mutations have been identified in AOP2 (e.g., residue 122) which are believed to alter its function. These studies are important for at least two reasons. First, they provide a reasonable expectation that still other homologues, allelic variants and mutants of this gene may exist in related species, such as dog, rat, rabbit, monkey, gibbon, chimp, ape, baboon, cow, pig, horse, sheep and cat. Upon isolation of these homologues, variants and mutants, and in conjunction with other analyses, certain active or functional domains can be identified. Second, this will provide a starting point for further mutational analysis of the molecule. One way in which this information can be exploited is in “domain switching.”

Domain switching involves the generation of chimeric molecules using different but, in this case, related polypeptides. By comparing the mouse and human sequences for Aop2 with the Aop2 of other species, and with mutants and allelic variants of these polypeptides, one can make predictions as to the functionally significant regions of these molecules. It is possible, then, to switch related domains of these molecules in an effort to determine the criticality of these regions to AOP2 function. These molecules may have additional value in that these “chimeras” can be distinguished from natural molecules, while possibly providing the same function.

Based on the sequence identity, at the amino acid level, of the mouse, dog and human sequences, it may be inferred that even small changes in the primary sequence of the molecule will affect function. Further analysis of mutations and their predicted effect on secondary structure will add to this understanding.

### E. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

### F. Purification of Proteins

It will be desirable to purify AOP2 or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS-PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS-PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

### G. Synthetic Peptides

The present invention also describes smaller AOP2-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

### H. Antigen Compositions

The present invention also provides for the use of AOP2 proteins or peptides as antigens for the immunization of animals relating to the production of antibodies. It is envisioned that either AOP2, or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyanin (KLH) or bovine serum albumin (BSA).

### III. Nucleic Acids

The present invention also provides, in another embodiment, genes encoding AOP2. Genes for the human and murine AOP2 molecule have been identified. The present invention is not limited in scope to these genes, however, as one of ordinary skill in the art could, using these two nucleic acids, readily identify related homologues in various other species (e.g., dog, rat, rabbit, monkey, gibbon, chimp, ape, baboon, cow, pig, horse, sheep, cat and other species). The finding of a mouse homologue for this gene makes it likely that other species more closely related to humans will, in fact, have a homologue as well. These gene may be used as part of diagnostic and therapeutic regimens.

In addition, it should be clear that the present invention is not limited to the specific nucleic acids disclosed herein. As discussed below, “an Aop2 gene” may contain a variety of different bases and yet still produce a corresponding polypeptides that is functionally indistinguishable, and in some cases structurally, from the human and mouse genes disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the function of Aop2.

### A. Nucleic Acids Encoding Aop2

The human gene disclosed in SEQ ID NO: 1 and the murine gene disclosed in SEQ ID NO:3 are Aop2 genes of the present invention. Nucleic acids according to the present invention may encode an entire AOP2 product, a domain of AOP2, or any other fragment of AOP2 sequences set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given Aop2 gene from a given species may be represented by natural variants that have slightly different nucleic acid sequences but, nonetheless, encode the same protein (see Table 1 below).

As used in this application, the term “a nucleic acid encoding an Aop2” refers to a nucleic acid molecule that has been isolated free of total cellular nucleic acid. In preferred embodiments, the invention concerns a nucleic acid sequence essentially as set forth in SEQ ID NO:1 or SEQ ID NO:3. The term “as set forth in SEQ ID NO:1 or SEQ ID NO:3” means that the nucleic acid sequence substantially corresponds to a portion of SEQ ID NO:1 or SEQ ID NO:3. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO: 1 or SEQ ID NO:3 will be sequences that are “as set forth in SEQ ID NO:1 or SEQ ID NO:3.” Sequences that are essentially the same as those set forth in FIG. X may also be functionally defined as sequences that are capable of hybridizing to a nucleic acid segment containing the complement of SEQ ID NO: 1 or SEQ ID NO:3 under standard conditions.

The DNA segments of the present invention include those encoding biologically functional equivalent AOP2 proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

### B. Oligonucleotide Probes and Primers

Naturally, the present invention also encompasses DNA segments that are complementary, or essentially complementary, to the sequence set forth in SEQ ID NO: 1 or SEQ ID NO:3. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 or SEQ ID NO:3 under relatively stringent conditions such as those described herein. Such sequences may encode the entire Aop2 protein or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3431 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to Aop2 or, more particularly, homologues of Aop2 from other species. The existence of a murine homologue strongly suggests that other homologues of the human Aop2 will be discovered in species more closely related, and perhaps more remote, than mouse. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double-stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

### C. Antisense Constructs

In some cases, mutant proteins may not be non-functional. Rather, they may have aberrant functions that cannot be overcome by replacement gene therapy, even where the “wild-type” molecule is expressed in amounts in excess of the mutant polypeptide. Antisense treatments are one way of addressing this situation. Antisense technology also may be used to “knock-out” function of AOP2 in the development of cell lines or transience mice for research, diagnostic and screening purposes.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

### D. Ribozymes

Another approach for addressing the “dominant negative” mutants is through the use of ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

### E. Vectors for Cloning, Gene Transfer and Expression

Within certain embodiments expression vectors are employed to express the Aop2 polypeptide product, which can then be purified and, for example, be used to vaccinate animals to generate antisera or monoclonal antibody with which further studies may be conducted. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

### (i) Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In one embodiment, the expression construct will comprise regulatory sequences found, in nature, in operable relation to the Aop2 gene. In certain embodiments, these regulatory sequences are fused to a reporter or indicator gene, such as luciferase or lacZ. In other embodiments, these regulatory sequences are operably connected to the Aop2 gene, either as they would be found in nature or in a modified fashion.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several elements/promoters which may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
ENHANCER/PROMOTER
Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
e-fos
c-HA-ras
Insulin
Neural Cell Adhesion Molecule (NCAM)
α1-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV4O
Polyoma
Retroviruses
Papilloma Virus
Hepatitis B Virus
Human Immunodeficiency Virus
Cytomegalovirus
Gibbon Ape Leukemia Virus

TABLE 3

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV4O | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP7O | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |
| Insulin E Box | Glucose |

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

### (ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

### (iii) Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

### (iv) Delivery of Expression Vectors

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1 , the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1 -deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol and env genes but without the LTR and packaging components is constructed (Mann et al, 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al, 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al, 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al, 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and

MDCK cells. In addition, a host cell strain may be chosen that modulates the expression of the inserted sequences, or modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent T-cells.

Large scale suspension culture of mammalian cells in stirred tanks is a common method for production of recombinant proteins. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

The antibodies of the present invention are particularly useful for the isolation of antigens by immunoprecipitation. Immunoprecipitation involves the separation of the target antigen component from a complex mixture, and is used to discriminate or isolate minute amounts of protein. For the isolation of membrane proteins cells must be solubilized into detergent micelles. Nonionic salts are preferred, since other agents such as bile salts, precipitate at acid pH or in the presence of bivalent cations. Antibodies are and their uses are discussed further, below.

### IV. Generating Antibodies Reactive With AOP2

In another aspect, the present invention contemplates an antibody that is immunoreactive with a AOP2 molecule of the present invention, or any portion thereof. An antibody can be a polyclonal or a monoclonal antibody. In a preferred embodiment, an antibody is a monoclonal antibody. Means for preparing and characterizing antibodies are well known in the art (see, e.g., Howell and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to AOP2-related antigen epitopes. Additionally, it is proposed that monoclonal antibodies specific to the particular AOP2 of different species may be utilized in other useful applications In general, both polyclonal and monoclonal antibodies against AOP2 may be used in a variety of embodiments. For example, they may be employed in antibody cloning protocols to obtain cDNAs or genes encoding other AOP2. They may also be used in inhibition studies to analyze the effects of AOP2-related peptides in cells or animals. Anti-AOP2 antibodies will also be useful in immunolocalization studies to analyze the distribution of AOP2 during various cellular events, for example, to determine the cellular or tissue-specific distribution of AOP2 polypeptides under different points in the cell cycle. A particularly useful application of such antibodies is in purifying native or recombinant AOP2, for example, using an antibody affinity column. The operation of all such immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are give in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified AOP2 protein, polypeptide or peptide or cell expressing high levels of AOP2. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10^{-8.} However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### V. Diagnosing Disease States Involving AOP2

The present inventors have determined that alterations in AOP2 and the related gene are associated with atherosclerosis. Therefore, AOP2 and the corresponding gene may be employed as a diagnostic or prognostic indicator of atherosclerosis and related disease states. More specifically, point mutations, deletions, insertions or regulatory pertubations relating to AOP2 may cause or promote atherosclerosis or related diseases.

### A. Genetic Diagnosis

One embodiment of the present invention comprises a method for detecting variation in the expression of AOP2. This may comprise determining the level of AOP2 expressed in cells or determining specific alterations in the expressed product. Obviously, this sort of assay has importance in the diagnosis of atherosclerosis and related diseases. Related disease states include coronary heart disease, ischemic heart disease and stroke.

The biological sample can be any tissue or fluid. Various embodiments include cells of the heart, blood circulatory system (arteries, veins endothelial lining of any vessel), skin, muscle, facia, brain, prostate, breast, endometrium, lung, head & neck, pancreas, small intestine, blood cells, liver, testes, ovaries, colon, skin, stomach, esophagus, spleen, lymph node, bone marrow, kidney or immune cells (e.g., monocytes, macrophages). Other embodiments include fluid samples such as peripheral blood, lymph fluid, ascites, serous fluid, pleural effusion, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have AOP2-related pathologies. In this way, it is possible to correlate the amount or kind of AOP2 detected with various clinical states or predisposition to clinical states.

Various types of defects are to be identified. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of AOP2 produced, both by altering the transcription of the gene or in destabilizing or otherwise altering the processing of either the transcript (mRNA) or protein.

A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR-SSCP.

### (i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹⁴C, ³⁵S, ³H, or other label), with a fluorophore (rhodamine, fluorescein) or a chemillumiscent (luciferase).

### (ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (” LCR“), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention, Walker et al. (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double-stranded DNA molecules are heat denatured again. In either case the single-stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single-stranded DNA, which is then converted to double-stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow”fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA” ) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: PCR PROTOCOLS: A GUIDE TO METHODS AND APPLICATIONS, Academic Press, N.Y., 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.

### (iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting of RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

### (iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

### (v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the Aop2 gene that may then be analyzed by direct sequencing.

### (vi) Kit Components

All the essential materials and reagents required for detecting and sequencing Aop2 and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

### (vii) Design and Theoretical Considerations for Relative Quantitative RT-PCR

Reverse transcription (RT) of RNA to cDNA followed by relative quantitative PCR (RT-PCR) can be used to determine the relative concentrations of specific mRNA species isolated from patients. By determining that the concentration of a specific mRNA species varies, it is shown that the gene encoding the specific mRNA species is differentially expressed.

In PCR, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

The second condition that must be met for an RT-PCR experiment to successfully determine the relative abundances of a particular mRNA species is that relative concentrations of the amplifiable cDNAs must be normalized to some independent standard. The goal of an RT-PCR experiment is to determine the abundance of a particular mRNA species relative to the average abundance of all mRNA species in the sample. In the experiments described below, mRNAs for B-actin, asparagine synthetase and lipocortin II were used as external and internal standards to which the relative abundance of other mRNAs are compared.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

One reason for this advantage is that without the internal standard/competitor, all of the reagents can be converted into a single PCR product in the linear range of the amplification curve, thus increasing the sensitivity of the assay. Another reason is that with only one PCR product, display of the product on an electrophoretic gel or another display method becomes less complex, has less background and is easier to interpret.

### (viii) Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994); Fodor et al. (1991).

### B. Immunodiagnosis

Antibodies of the present invention can be used in characterizing the AOP2 content of healthy and diseased tissues, through techniques such as ELISAs and Western blotting. This may provide a screen for the presence or absence of antioxidant activity or as a predictor of atherosclerosis.

The use of antibodies of the present invention, in an ELISA assay is contemplated. For example, anti-AOP2 antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for AOP2 that differs from the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

### VI. Methods for Screening Active Compounds

The present invention also contemplates the use of AOP2 and active fragments, and nucleic acids coding therefor, in the screening of compounds for activity in either stimulating AOP2 activity, overcoming the lack of AOP2 or blocking the negative effects of a mutant AOP2 molecule. These assays may make use of a variety of different formats and may depend on the kind of “activity” for which the screen is being conducted. Activity may be assayed in a cell-free system, and generally will comprise monitoring oxidation or prevention of oxidation of proteins or lipids. Specifically, one will measure protection of glutamine synthase from inactivation after addition of thiol-containing reducing agents (e.g., DTT) and metal (e.g., Fe⁺³). Activity also can be measured in a cell-free system by virtue of AOP2's ability to block O₂ consumption when incubated with thiol-containing reducing agent and metal ion (Netto et al., 1996).

### A. In Vitro Assays

In one embodiment, the invention is to be applied for the screening of compounds that bind to the AOP2 molecule or fragment thereof. The polypeptide or fragment may be either free in solution, fixed to a support, expressed in or on the surface of a cell. Either the polypeptide or the compound may be labeled, thereby permitting determining of binding.

In another embodiment, the assay may measure the inhibition of binding of AOP2 to a natural or artificial substrate or binding partner. Competitive binding assays can be performed in which one of the agents (AOP2, binding partner or compound) is labeled. Usually, the polypeptide will be the labeled species. One may measure the amount of free label versus bound label to determine binding or inhibition of binding.

Another technique for high throughput screening of compounds is described in WO 84/03564. Large numbers of small peptide test compounds are synthesized on a solid substrate, such as plastic pins or some other surface. The peptide test compounds are reacted with AOP2 and washed. Bound polypeptide is detected by various methods.

Purified AOP2 can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the AOP2 active region to a solid phase.

Various cell lines containing wild-type or natural or engineered mutations in AOP2 can be used to study various functional attributes of AOP2 and how a candidate compound affects these attributes. Methods for engineering mutations are described elsewhere in this document, as are naturally-occurring mutations in AOP2. In such assays, the compound would be formulated appropriately, given its biochemical nature, and contacted with a target cell. Depending on the assay, culture may be required. The cell may then be examined by virtue of a number of different physiologic assays. Alternatively, molecular analysis may be performed in which the function of AOP2, or related pathways, may be explored. This may involve assays such as those for protein expression, enzyme function, substrate utilization, phosphorylation states of various molecules including AOP2, cAMP levels, mRNA expression (including differential display of whole cell or polyA RNA) and others.

One also may employ constructs where reporter genes are linked to Aop2 coding sequences or regulatory sequences. In this way, the increase in expression may be measured by looking at a distinct function, associated with the reporter, that is easier to measure than, for example, antioxidant activity. Examples of reporter genes include CAT, β-galactosidase, luciferase and green fluorescent protein.

### B. In Vivo Assays

The present invention also encompasses the use of various animal models. Here, the high degree of homology seen between human and mouse AOP2 provides an excellent opportunity to examine the function of AOP2 in a whole animal system where it is normally expressed. By developing or isolating mutant cells lines that fail to express normal AOP2, one can generate animal models that will be highly predictive of diseases, including atherosclerosis, in humans and other mammals. Finally, transience or congenic animals (discussed below) that lack a wild-type AOP2 may be utilized as models for atherosclerosis development and treatment.

Treatment of animals with test compounds will involve the administration of the compound, in an appropriate form, to the animal. Administration will be by any route that can be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, increased activity level, reduced oxidation of protein or lipid, lowered HDL level, reduced oxidative products, reduced atherosclerosis, reduced oxidized LDL, reduced aging and reduced brain pathology.

### C. Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration or which may affect the function of various other molecules. By virtue of the availability of cloned Aop2 sequences, sufficient amounts of AOP2 can be produced to perform crystallographic studies. In addition, knowledge of the polypeptide sequences permits computer employed predictions of structure function relationships. In one approach, one would generate a three-dimensional structure for AOP2 or a fragment thereof. This could be accomplished by x-ray crystallography, computer modeling or by a combination of both approaches. An alternative approach, “alanine scan,” involves the random replacement of residues throughout molecule with alanine, and the resulting affect on function determined.

It also is possible to isolate a AOP2-specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved AOP2 activity or which act as stimulators, inhibitors, agonists, antagonists of AOP2 or molecules affected by AOP2 function. Another drug design approach relates to the functional aspects of AOP2, namely, its putative role as an antioxidant. Using compounds that are antioxidants, that promote antioxidant activity or that prevent oxidate directly, one may design new compounds that function in conjunction with AOP2.

### VII. Methods for Treating AOP2 Related Disease States

The present invention also involves, in another embodiment, the treatment of atherosclerosis. This treatment may comprise provision of AOP2 or provision of an expression construct containing an Aop2 gene.

### A. Genetic Based Therapies

One of the therapeutic embodiments contemplated by the present inventors is the intervention, at the molecular level, in the events involved in the development of atherosclerosis. Specifically, the present inventors intend to provide, to an appropriate target cell, an expression construct capable of providing AOP2 to that cell. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient or animal. Similar figures may be extrapolated for liposomal or other non-viral formulations by comparing relative uptake efficiencies. Formulation as a pharmaceutically acceptable composition is discussed below.

### B. Protein Therapy

Another therapy approach is the provision, to a subject, of AOP2 polypeptide, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

### C. Combined Therapy with Traditional Anti-Atherosclerotic Therapy

One goal of current research is to find ways to improve the efficacy of pharmaceuticals that are designed to reduce atherosclerosis. One way is by combining such traditional therapies with gene therapy. In the context of the present invention, it is contemplated that Aop2 replacement therapy could be used similarly in conjunction with traditional pharmaceutical intervention.

Generally, one will contact a “target” cell with an Aop2 expression construct and at least one other agent. These compositions would be provided in a combined amount effective to reduce the atherosclerotic burden of the animal. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an Aop2 expression construct or the other agent will be desired. Various combinations may be employed, where Aop2 is “A” and the other agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated.

Agents or factors suitable for use in a combined therapy are any chemical compound or treatment method that reduces the atherosclerotic burden on a host or alters cholesterol or lipoprotein levels. The agent may be prepared and used as a combined therapeutic composition, or kit, by combining it with a Aop2 expression construct, as described above.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the regional delivery of Aop2 expression constructs to patients with AOP2-linked diseases will be a very efficient method for delivering a therapeutically effective gene to counteract the pathological disease. Similarly, the therapy may be directed to a particular, affected region of the subject's body. Alternatively, systemic delivery of expression construct and/or the agent may be appropriate in certain circumstances occurred.

### D. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VIII. Congenic, Transgenic and Knockout Animals

In one embodiment of the invention, congenic animals are produced that have “normal” or mutant Aop2 genes. These animals will find use in testing of compounds in vivo that stimulate or inhibit AOP2 expression or function. For example, compounds that improve the antioxidant activity of AOP2 can be identified using these congenic animals, using a variety of different functional readouts, as described above. Methods for breeding congenic strains are well known to those of skill in the art.

In another embodiment, transgenic animals are produced which contain a functional or non-functional (e.g., mutant) transgene encoding a functional AOP2 polypeptide or variants thereof. Transgenic animals expressing Aop2 transgenes, recombinant cell lines derived from such animals and transgenic embryos may be useful in methods for screening for and identifying agents that induce or repress function of AOP2. Transgenic animals of the present invention also can be used as models for studying indications such as atherosclerosis.

In one embodiment of the invention, an Aop2 transgene is introduced into a non-human host to produce a transgenic animal expressing a human or murine Aop2 gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous Aop2 by homologous recombination between the transgene and the endogenous gene; or the endogenous gene may be eliminated by deletion as in the preparation of “knock-out” animals. Typically, an Aop2 gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which overexpress AOP2 or express a mutant form of the polypeptide. Alternatively, the absence of a Aop2 in “knock-out” mice permits the study of the effects that loss of AOP2 protein has on a cell in vivo. Knock-out mice also provide a model for the development of AOP2-related diseases.

As noted above, transgenic animals and cell lines derived from such animals may find use in certain testing experiments. In this regard, transgenic animals and cell lines capable of expressing wild-type or mutant AOP2 may be exposed to test substances. These test substances can be screened for the ability to enhance wild-type AOP2 expression and or function or impair the expression or function of mutant AOP2.

### IX. EXAMPLES

The following examples are included to demonstrate preferred embodiments of the invention. It should be appreciated by those of skilled the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1—Materials and Methods

Mice. C57BL/6J, DBA/2J, and 8 BXD recombinant inbred strains (BXD 1, BXD 2, BXD 5, BXD 6, BXD 8, BXD 9, BXD 11, BXD 25) were obtained from The Jackson Laboratory (Bar Harbor, Maine).

Two dimensional gel electrophoresis: Proteins were extracted from kidney and liver by a modification of the method of Wilson et al. (1977). 200 mg of tissue were homogenized on ice in 68 μl of lysis buffer containing 10% mercaptoethanol, 1% SDS, 8 M urea. Five hundred μl of boiling sample buffer 1 (0.3% SDS, 200 mM DTT, 28 mM Tris HCl, 22 mM Tris-base) was added followed by incubation at 100° C. for 5 min. Samples were chilled on ice for 5 min. mixed with 50 μl of sample buffer 2 (24 mM Tris-base, 476 mM Tris-HCl, 50 mM MgCl2, 1 mg/ml DNAase I, 25 mg/ml RNAse A), incubated on ice for 8 min. and proteins were precipitated with 80% acetone at 0° C. for 20 min. The suspension was microcentrifuiged (10,000 g, 5 min.) and the precipitate was resuspended in 500 μl of loading buffer (7.92 M urea, 0.06% SDS, 1.76% Ampholytes solution pH 3-7, 120 mM DTT, 3.2% Triton X-100, 22.4 mM Tris HCl and 17.6 mM Tris-base). Fifteen μl of each sample (approximately 10 μg of protein) was electrophoresed. Analytical and preparative two-dimensional electrophoresis were performed using apparatus, protocols and 2-D grade reagents purchased from the Millipore Corp. Isoelectric point and molecular weight standards (BioRad) were run simultaneously with analyzed protein samples.

Protein blotting and protein microsequencing. Proteins were transferred from preparative two-dimensional electrophoresis gels using the MilliBlot Graphite Semi-Dry Electroblotter System and Immobilon-P (PVDF) Transfer Membrane (Millipore) in 0.5×Tris-Glycine Towbin buffer according to protocol provided by manufacturer. After transfer the membrane was stained with 0.25% Coomassie brilliant blue, and the protein spot was identified and excised from dry membrane. N-terminal amino acid sequence was obtained using an Applied Biosystems Model 477A protein sequencer, modified as described by Tempst and Riviere²⁴. Homology searches were performed against the Genpet and dbEST databases. Sequence alignment and consensus analysis of the EST sequences was done using Sequencher 3.0 (Gene Codes). Genbank accession numbers for ESTs used for sequence alignment include: W70913, W71818, W14400, W51064, W59407, W83606, W83464, AA002970. Terminal 3′ UTR sequence was obtained using Sequenase (USB) according to the manufacturer's instructions.

SSCP mapping. Single-stranded conformational polymorphism (SSCP) analysis of the 3′ UTR of the presumptive Ltw4 gene was employed for genetic mapping (Guertin et al., 1994; Hahn & Subbiah, 1994). A polymorphism between the C57BL/6J and M. spretus strains was identified using PCR amplification of the primers:

5′-GCGAGCGATCTACAGGACC-3′ (SEQ ID NO: 9)
and 5′-TGATGGTAGTTCCCACCCTC-3′ (SEQ ID NO: 10)

Mapping was done using the panel of 94 DNAs from the BSS interspecific backcross (lakoubova et al., 1997) as follows: 30 ng of DNA was amplified in 96 wells microtiter plates with ³²P end-labeled forward and reverse primers (0.3 μM of each primer) in total volume of 12 μl of PCR buffer containing 10 mM Tris pH 8.0, 50 mM KCL, 2 mM MgCl₂ and 0.001% gelatin. After denaturation at 94° C. for 5 min. 0.2 mM dNTPs and 0.5 units of Taq DNA polymerase were added. Twenty-five cycles of amplification (55° C. annealing for 2 min. 72° C. extension for 3 min. 94° C. denaturation for 1 min.) were performed with a final extension step of 7 min. PCR products were mixed 1:4 with USB stop solution, denatured at 95° C., transferred on ice and 2 μl of each sample electrophoresed through a 5% non-denaturing polyacrylamide gel in 0.5×TBE buffer at 4° C., 40 W constant power for 2-3 hr. The strain distribution pattern was analyzed using the Map Manager computer program.

### Example 2—Results

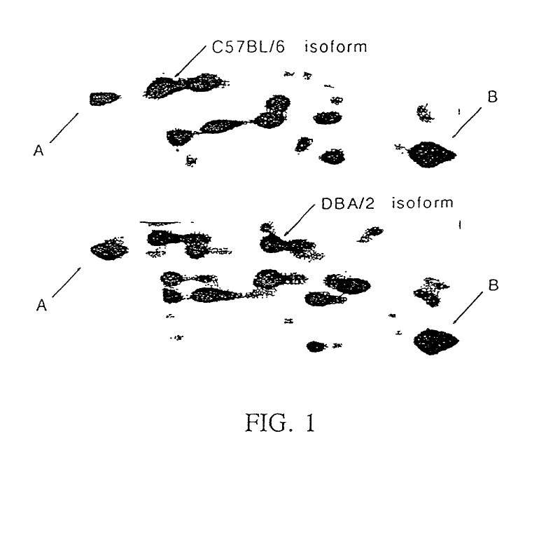

LTW4 protein was identified by surveying approximately one thousand silver-stained protein spots obtained after two-dimensional electrophoresis of liver and kidney homogenates for a polymorphic protein similar to that originally described by Elliott, 1979. A polypeptide of 26 kD was identified whose C57BL/6J allele had a pI of 5.9 and whose DBA/2J allele had a pI of 5.6 (FIG. 1). To ensure that the polymorphic protein identified corresponded to LTW4, 8 BXD RI strains were analyzed using two-dimensional gel electrophoresis, and determined that the strain distribution pattern of this protein in these strains was identical to that described for Ltw4.

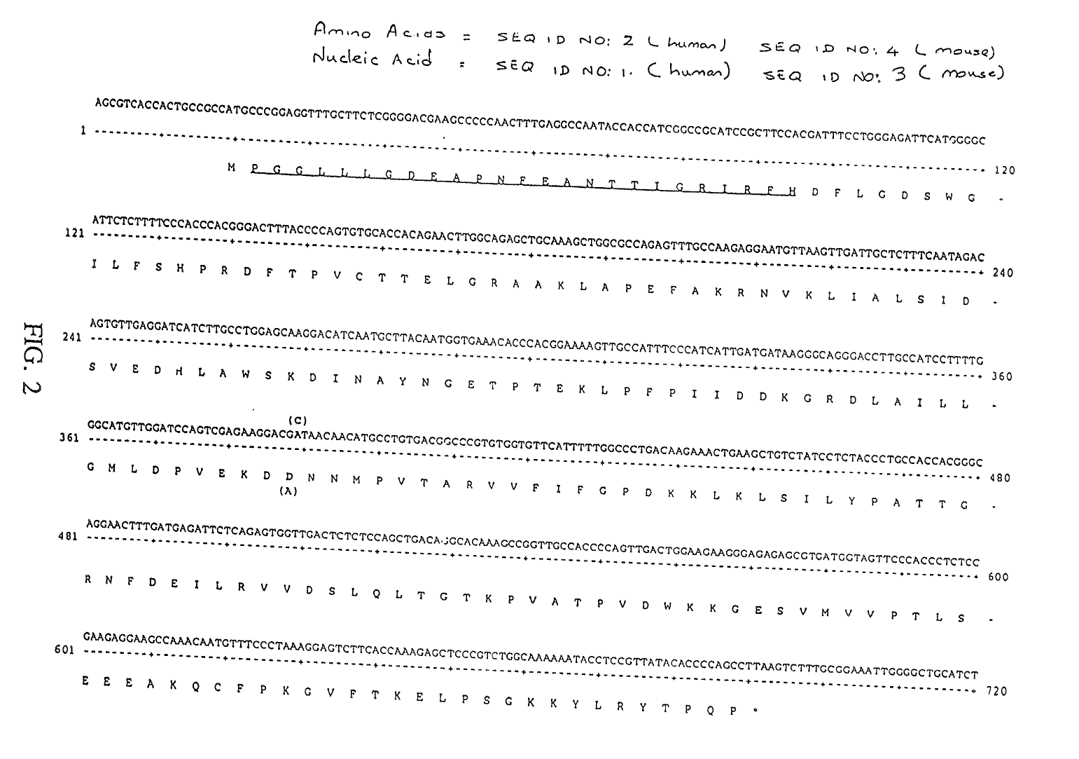

Preparative 2-D electrophoresis was performed using liver proteins extracted from DBA/2J strain, since this variant of LTW4 was more readily resolved from other proteins than the C57BL/6J-related isoform. After electrophoresis in the second dimension, proteins were transferred to a membrane and the LTW4 protein spot identified and isolated. Microsequencing of the protein spot yielded twenty-four N-terminal amino acids that had 92% identity with residues 2-26 of a previously identified human hypothetical protein (HUMORF06, Genbank accession number: D14662) which appears to be a member of a large family of thiol-specific antioxidant (TSA) proteins (Elliott et al., 1980). The nucleotide sequence of HUMORF06 was used to identify homologous murine partial cDNA sequences using BLASTN searches against the dbEST database. A consensus sequence from these ESTs was obtained using Sequencher 3.0 sequence alignment software (FIG. 2). The translated product of this concensus sequence has 89% identity and 93% similarity with HUMORF06. As discussed below, another TSA family member, the murine MER5 gene, recently renamed antioxidant protein 1 (Aop1), has regions of conserved homology with the gene characterized here; therefore, this gene has been designated antioxidant protein 2 (Aop2).

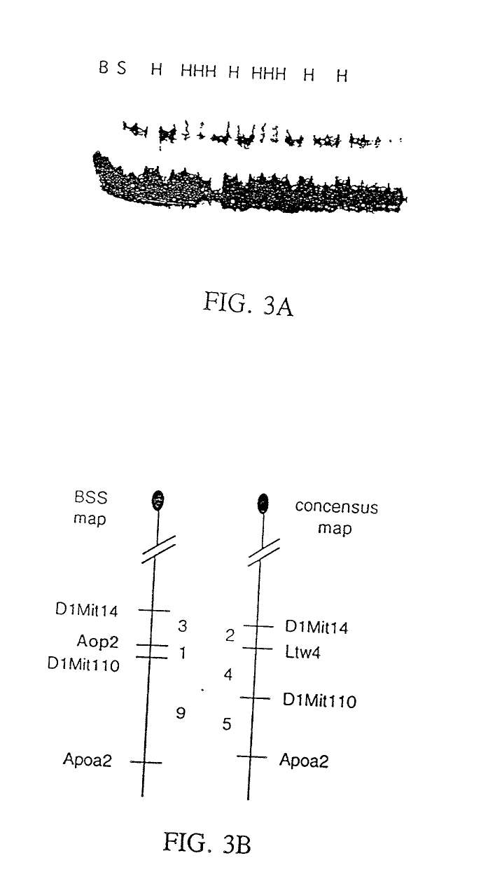

SSCP analysis was used as previously described to assess whether the gene encoding the polymorphic protein isolated maps to the same position as Ltw4 (Guertin et al., 1994; Hahn & Subbiah, 1994). An SSCP between C57BL/6J and M. spretus was identified using primers derived from 3′ UTR of Aop2 and these primers were then tested in the BSS interspecific backeross (FIG. 3A & B) (Iakoubova et al., 1997). Aop2 was localized to chromosome 1 with a LOD likelihood of 27.4. No recombinants were observed between Aop2 and Pmx1 in 91 backcross progeny, and the position of these loci with respect to flanking microsatellite markers is D1Mit14−3.2±1.8 cM-(Aop2, Pmx1) −2.2±1.5 cM-D1Mit110. The map position of Ltw4 in the BXD RI series is D1Mit111 −23±11 cM−(Ltw4,At3)−3.1±3.2 cM−D1Mit16. Since D1Mit16 is 1.1 cM proximal to D1Mit110, the position of Aop2 and Ltw4 with respect to these distal Mit microsatellite markers is within the same 95% confidence interval. In addition, At3, which is nonrecombinant with Ltw4 in the BXD series, maps between D1Mit 14 and D1 Mit110 (Marcel et al., 1989). Finally, the region of chromosome 1 in which Ltw4 has been mapped demonstrates conservation of synteny with human chromosome 1q23-25. WI-7237, an STS derived from HUMORF06 (the human homologue of Aop2), has been mapped in this region on radiation hybrid and YAC physical maps (Ohkawa et al., 1979). These mapping studies confirm that Aop2 corresponds to Ltw4.

### Example 3—Discussion

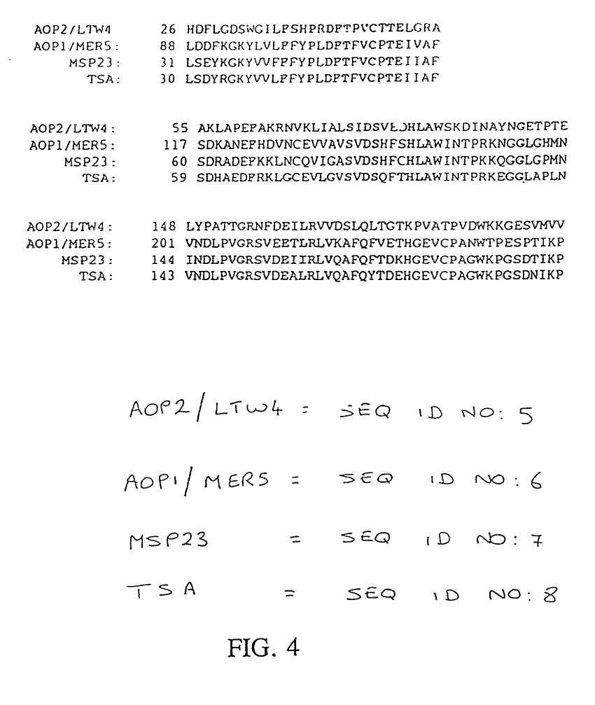

LTW4 protein shares amino acid similarity with members of a class of thiol-specific antioxidant (TSA) proteins that have been identified in a wide variety of organisms (Elliott et al., 1980). The best characterized of these proteins include yeast TSA, which has been shown to protect cellular components against oxidative damage from a system capable of generating reactive sulfur species, but not from a system that generates only reactive oxygen (Paigen et al., 1987a). This activity is also found for AhpC, the catalytic component of the alkyl hydroperoxide reductase from S. typhimurium (also called C22) (Elliott et al., 1980). The mammalian members of this protein family include brain TSA (Elliott et al., 1980), ER5 (Paigen et al., 1987b), osteoblast-specific factor 3 (OSF3) (Paigen et al., 1987c), natural killer enhancer factors A and B (NKEFA and NKEFB) (Paigen et al., 1990), proliferation-associated gene (PAG) (Parthasarathy et al., 1990), and macrophage 23 kD stress protein (MSP23) (Senault et al., 1990). (OSF3, NKEFA, PAG and MSP23 are highly homologous and may be variants of the same protein.) Amino acid comparison of AOP2, MER5, MSP23, and TSA using the Blocks program (http://www.blocks.fhcrc.org) reveals three domains of conservation (FIG. 4). It is apparent that AOP2 is the least similar of these proteins; of particular interest is that AOP2 does not share the second cysteine that is conserved in the other three murine proteins as well as in most members of the TSA gene family (Elliott et al., 1980). While the specific functions of these genes have not yet been elucidated, it is of note that MSP23 was initially isolated as a protein that was induced in response to oxidative stress. In addition, it has been shown that the gene encoding MER5 can complement a deficiency of alkyl hydroperoxide reductase activity in E. coli that is due to a mutation in AhpC (Yamamoto et al., 1989). Based upon this result, the Mer5 gene has been renamed antioxidant protein 1 (Aop1).

The protein encoded by Aop2 was originally found to be an abundant product in homogenates of liver, kidney, and brain (Chae et al., 1994; Elliott, 1979). Aop2 cDNA appears to be widely expressed: the TIGR database (http://www.tigr.org/tdb/tdb.html) identifies a tentative human concensus (THC122873) that corresponds to HUMORF06 (the human homologue of Aop2), this THC is comprised of 116 ESTs that have been identified in cDNA libraries derived from 24 different tissues.

### Example 4—Materials and Methods

SSCP mapping. Single-stranded conformational polymorphism (SSCP) analysis of the 3′ untranslated region of the Aop2 gene was employed for genetic mapping in the congenic Athl strains (Beier et al., 1992; Beier, 1993). A polymorphism between the C57BL/6J and M. spretus strains was identified using PCR amplification of the primers:

5′-GCGAGCGATCTACAGGACC-3′ (SEQ ID NO: 9)
and 5′-TGATGGTAGTTCCCACCCTC-3′ (SEQ ID NO: 1O)

Mapping was performed using the BSS Jackson Laboratory panel of backcross mice derived from backcross of (C57BL/6 X Spretus) F1 mice backcrossed to Spretus as follows: 30 ng of DNA was amplified in 96-well microtiter plates with 0.3 mM ³²P end-labeled forward and reserve primers in a total volume of 12 ml of PCR buffer containing 10 mM Tris pH 8.0, 50 mM KCL, 2mM MgCl₂ and 0.001% gelatin. After denaturation at 94° C. for 5 min., 0.2 mM dNTPs and 0.5 units of Taq DNA polymerase were added. Twenty-five cycles of amplification (55° C. annealing for 2 min, 72° C. extension for 3 min, 94° C. denaturation for 1 min) were performed with a final extension step of 7 min. PCR products were mixed 1:4 with USB stop solution, denatured at 95° C., transferred to ice and 2 ml of each sample electrophoresed through a 5% non-denaturing polyacrylamide gel in 0.5×TBE buffer at 4° C., 40 W constant power for 2-3 hr. Using this approach, mice heterozygous for C57BL/6J and M. spretus alleles could be distinguished from mice homozygous for the C57BL/6J allele. Homozygosity for the C57BL/6J allele of Aop2 and susceptibility to atherosclerosis were concordant in 100% of these mice. This corresponds to a genetic distance of 0-x cM.

Sequence analysis: Clones containing full-length open reading frames were obtained using RT-PCR amplification from cDNA prepared from kidney and liver of C57BL/6J, DBA/2J, and C3H/FeJ mice according to standard techniques. Primers used for this amplification were AOP2 orf forward: 5′-AGCGTCACCACTGCCGCCATG-3′ (SEQ ID NO:11) and AOP2 orf reverse: 5′-GTACTGGATGTGCAGATGCAGCC-3′ (SEQ ID NO:12). Multiple independent PCR reactions were done for each strain and the amplified fragment cloned into pCR2.1 (Invitrogen) and sequenced using Sequenase (USB) according to the manufacturer's instructions.

### Example 5—Results

Construction of the congenic strain. In order to construct a congenic strain with susceptible C57BL/6 (B6) as the host strain, and the resistant allele of Athl from another strain with more polymorphic differences from B6 than the existing congenic B6.C-H25^{c}/By, a region of the Spretus genome was introduced into B6 as these two strains are from different, although closely related, species. The Athl region from Spretus was backcrossed into B6 using the markers D1Mit14 and D1Mit136 for 5 generations and then intercrossing to obtain the F1 generation. The attempt to breed the NSF1 generation to homozygosity failed; homozygous F2 mice were obtained but did not breed to N5F2. The F2 males apparently have a deficiency of sperm, and these sperm do not fertilize eggs even by in vitro fertilization. The heterozygote congenic is being preserved as frozen embryos.

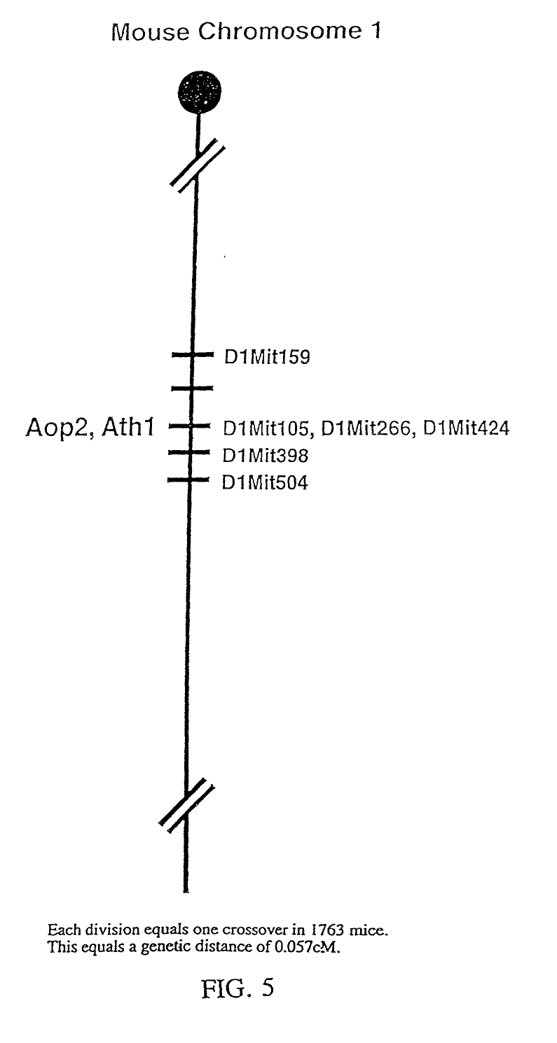

High resolution mapping of Athl. To map Athl with high resolution, a backcross was carried out between C57BL/6 and the heterozygous congenic strain B6.Spret-Athl^{r/s}. This cross included 1176 progeny, and 89 crossovers were obtained between the markers D1Mit14 and D1Mit356, which flank the Athl region. Mice carrying crossovers were mated to B6 and its female progeny collected and fed the high fat diet for 18 weeks. Lesion size was determined for each mouse and averaged over all progeny from each crossover parent to determine whether the parent carried the resistant or susceptible allele of Athl. Polymorphic genetic loci were typed in the crossover animals; the high resolution map is depicted in FIG. 5. Each crossover represents a genetic distance of 1763/100 or 0.057 cM.

No recombinant mice were found between Athl and the SSLP loci D1Mit105, D1Mit266, and D1Mit424. Since Aop2 mapped to this region of murine chromosome 1, primer pairs were utilized that recognize a polymorphic difference in the 3′ -untranslated region of Aop2. These were mapped in a panel of mice that carried unique crossovers in progeny of the high resolution backcross. No recombinants were found between Athl and Aop2, suggesting that Aop2 is Athl.

Sequencing Athl/Aop2 cDNA. Primers flanking the presumptive coding region of Athl/Aop2 were utilized to amplify and clone this region from kidney and liver cDNA prepared from the C57BL/6J, DBA/2J, and C3H/FeJ inbred mouse strains; the sequence of these clones appears in FIG. 2. The C57BL/6J and DBA/2J alleles of this gene code for proteins with a single amino acid difference: amino acid 122 corresponds to aspartic acid in DBA/2J and to alanine in C57BL/6J. The translated product of the full length coding sequence has 89% identity and 93% similarity with a human sequence HUMORF06. The protein encoded by Aop2 was originally found to be an abundant product in homogenates of liver, kidney, and brain (Elliott, 1979; Racine and Langley, 1980; Goldman and Pikus, 1986).

### Example 6—Discussion

Aop2 was initially characterized as a candidate for the Jckm2 modifier locus. The possible role of an antioxidant protein in the progression of polycystic kidney disease is not obvious. It is of note, however, that Aop2 maps in the region identified for atherosclerosis 1 (Athl), a locus that has been suggested to play a role in determining the difference in the relative susceptibility of the C57BL/6J and C3H/HeJ mouse strains to the development of atherosclerotic plaques when fed a high fat diet. The role of lipid oxidation in the pathogenesis of atherosclerosis is well known, and recent studies have implicated Athl in either the accumulation of lipid peroxides in tissues, or the cellular response to such lipid peroxides. Since the Aop2 protein product is polymorphic between C57BL/6J and C3H/HeJ, and since the biochemical functions of other members of this gene family include the reduction of peroxides (lakoubova et al., 1997), Aop2 should was considered the candidate for Athl. Aop2 cDNA appears to be widely expressed. The TIGR database (http://www.tigr.org/tdb/tdb.html) identifies a tentative human concensus (THC122873) that corresponds to HUMORF06. the human homolog of Aop2. This THC is comprised of 116 ESTs that have been identified in cDNA libraries derived from 24 different tissues.

### Example 7

As discussed above, Athl is associated with reduced plasma HDL levels and increased atherosclerotic lesion formation in susceptible mice when fed a high fat diet, as compared to Athl resistant mice which exhibit no change in plasma HDL levels and the absence of atherosclerotic lesions. The mapping of this locus to chromosome 1 cM 83.6 in the mouse genome coincided with the cloning and mapping of Aop2. Based on amino acid homology, Aop2 appears to encode a new member a large family of thiol-specific antioxidants (TSA), which are highly conserved (Chae et al., Proc. Natl. Acad. Sci. USA 91: 7017-7021 (1994)). These proteins possess activity which protects cells from oxidative damage resulting from metal-catalized oxidation systems, and are thus thought to be an important part of the complex cellular defense against oxidative stress. Aop2 cDNA was isolated from liver and kidney, but was also found to be expressed in a wide variety of tissues (lakoubova et al., Genomics 42: 474-478 (1997)). This gene was found to encode the previously characterized Ltw-4 protein, which was shown to be polymorphic between the C57BL/6J and DBA/2J strains of mice.

The cDNA corresponding to Aop2 has been independently isolated by two other laboratories, one of which has called it a glutathione peroxidase due to its high identity a bovine ciliary glutathione peroxidase (Frank et al., Oncogene 14: 915-921 (1997); Munz et al., J. Biochem. 326: 579-585 (1997)), and the other has called it a phospholipase for the presence of a “GXSXG” motif, which is reminiscent of serine hydrolase activity (Kim et al., J. Biol. Chem. 272: 2542-2550 (1997)). However, Aop2 shows no significant homology with any other known glutathione peroxidases, nor does it resemble any phospholipases that have been identified. In contrast, Aop2 demonstrates significant homology with over 50 known thiol-specific antioxidants. Furthermore, TSA activity has in fact been demonstrated for the human homolog of Aop2. Therefore, Aop2 appears to be a member of the TSA gene family. Since LDL oxidation has been implicated in foam cell formation and atherosclerotic lesion development in the arterial wall, Aop2 was investigated as a candidate gene for Athl.

The Athl locus was previously mapped to a 3 cM region on chromosome 1 using recombinant inbred strains. Since there were insufficient polymorphic markers between B6 and C3H, two independent congenics were made, which carried the Athl allele from the atherosclerosis-resistant wild-derived strains, Spretus and PERA, on a C57BL/6J background. Using a congenic on a B6 background avoids the problems of all the other genes in these wild-derived strains that affect HDL and atherosclerosis, but adds the benefit of many polymorphic markers in the region of Athl. Backcross progeny were collected from the N5 incipient congenic derived from Spretus; theoretically the congenic at the fifth generation has only about 3% of Spretus gene except for the selected region on Chromosome 1. Therefore, the chance that other Spretus genes besides Athl would affect HDL-C and lesions in a cross with B6 is greatly reduced. 1763 backcross progeny were collected from this cross and tested for progeny with recombination events between D1Mit14 and D1Mit356, a genetic distance of 10 cM based on the MIT map. A total of 162 recombinant mice were accumulated and analyzed in a two step process. Step 1: Female recombinants were tested for lesions, accepting the fact that one can not rely on the phenotype from each individual mouse due to the high variability in this quantitative trait. This narrowed the region to D1Mit14- -D1Mitl 10, a 3-4 cM region. Step 2: Male mice with a recombination event in this 3-4 cM region were bred, and female progeny tested for atherogenic phenotype. This progeny testing avoids the problem of high variability by testing groups of progeny mice to determine the phenotype of a single recombinant mouse. This two step procedure narrowed the region considerably. Each recombinant equals a distance of 1/1763 or 0.05 cM. If 1 cM equals 1.75 megabases, the physical distance between each crossover can be estimated to be about 87.5 kb. Genes are estimated to occur on the average every 40 kb (although there are gene rich and gene poor regions). A fine structure map of this region reveals that Athl shows no recombination with D1Mit105, D1Mit266 and D1Mit424, and lies in a region between D1Mit159 and D1Mit398 that is 0.15 cM in length. This excludes, as a candidate, apolipoprotein A2, whose map position is below this region. It also excludes Acact (acylcoenzyme A:cholesterol acytransferase), the gene coding for the enzyme ACAT, which metabolizes cholesterol to a storage molecule, cholesterol ester, which maps above this region.

Since it was previously shown that Aop2 is polymorphic between C57BL/6J and Spretus, the 37 mice that were recombinant in the Athl region were directly assayed for which Aop2 allele they carried. These results showed no recombination with Athl. Because an antioxidant protein is an attractive candidate gene for atherosclerosis, the next steps are to determine if Aop2 is in fact Athl.

In order to determine if resistant and susceptible phenotypes correlate with different Aop2 alleles, Aop2 cDNA was amplified and sequenced from the susceptible C57BL/6J strain, four atherosclerosis-resistant inbred strains (C3H/HeJ, BALB/cJ, DBA and 129/SvJ), and the two atherosclerosis-resistant wild strains, Spretus and PERA, used to independently derive the Athl congenics. The nucleotide sequences corresponding to the coding region of Aop2, and the corresponding amino acid sequences were determined in each strain. All four resistant inbred strains possessed the single amino acid difference (at amino acid#124) from the susceptible B6 strain. This amino acid is alanine in the susceptible C57BL/6J strain, and aspartic acid in each of the four resistant inbred strains. Although the functional significance of this amino acid difference is not known, prior analysis of this difference has revealed that it can account for the difference in the 2D gel migration pattern of Aop2 protein previously reported between the B6 and DBA strains. In contrast, the two atherosclerosis resistant wild strains that were analyzed, PERA and Spretus, do not possess this amino acid difference and appear to carry the same Aop2 allele as the susceptible C57BL/6J. Therefore, the mere presence of this amino acid difference could not explain resistance in all strains. For this reason, the characterization of Aop2 mRNA expression was undertaken to determine if these strains exhibited differences in Aop2 expression.

Aop2 mRNA levels were compared from liver tissue of the susceptible B6 strain, the resistant BALB/cJ strain, and the two resistant Athl congenic strains, B6. PERA and B6. Spretus, on both chow or high fat diet for 4 or 10 weeks. Total RNA was isolated from individual livers, and RNA from two individual mice, and a pool of 10 mice were analyzed from each strain on both chow or high fat diet. Aop2 expression in the liver was similar on chow and high fat diet in those resistant strains possessing the Aop2 allele carrying aspartic acid at amino acid position 124. In contrast, all strains carrying the Aop2 allele encoding alanine at amino acid position 124 showed a significant induction of Aop2 on high fat diet. Induction occurred in all three strains by 4 weeks on diet. However, the level of Aop2 expression on both chow and high fat diet was significantly greater in the resistant strains, as compared to the susceptible C57BL/6J.

To further support the conclusion that Aop2 is Athl, a correlation between Athl phenotype and Aop2 expression in critical crossover animals put on high fat diet was investigated. The presence or absence of atherosclerotic lesions in these individuals was compared with their Aop2 expression in the liver. The two resistant B6. Spretus congenic animals (N6) showed high levels of Aop2 expression after 14 weeks on high fat diet. These animals did not get lesions. In addition, 4 crossover animals, which lacked lesions, and four which developed atherosclerotic lesions by 14 weeks on high fat diet, were directly compared. Those animals which were resistant to lesions expressed higher levels of Aop2 after 14 weeks on high fat diet than did those which developed lesions. Therefore, there is a correlation between development of atherosclerotic lesions and expression level of Aop2.

### Example 8—Materials & Methods

### (a) Isolation and Subcloning of Aop2 Genomic Clones.

To isolate a genomic clone for the mouse Aop2 gene, a 0.7 kb cDNA fragment spanning the entire coding region of the murine Aop2 cDNA was amplified from the DBA mouse strain by PCR using Aop2orf forward (5′-AGCGTCACCACTGCCGCCATG-3′) and Aop2orf reverse (5′-GTACTGGATGTGCAGATGCAGCC-3′) primers. This fragment was isolated, 32P-radiolabeled by random priming (Amersham, RediPrime Kit), and used as a probe to screen a 129/SvJ IFIXRII genomic library (Stratagene, La Jolla, Calif., USA). Approximately 1.8×10⁶ plaques were screened. Individual positive clones were selected after a second round of screening, and phage DNA was isolated from each clone using standard techniques. Southern blotting was performed on EcoRI digested genomic clones using the ³²P-radiolabeled Aop2 cDNA probe to distinguish unique sequences. Preliminary sequencing was performed on lambda clones using the dideoxy chain termination method with both the M13 forward and reverse primers, and specific primers designed according to the published Aop2 cDNA sequence (lakoubova et al, Genomics 42: 474-478 (1997)). Positive clones were digested with NotI, subcloned into the pBluescript SK—vector (Stratagene, La Jolla, Calif., USA) and were sequenced using the dideoxy chain termination method (USB).

### (b) Determination of Intron/Exon Junctions, Intron Size, & Promoter Sequence

The intron/exon junctions of Aop2 were determined by sequencing of subcloned genomic fragments using exon-specific primers flanking each intron. These included: Exon1F (5′GAGGATTGCTTCTCGGGG-3′), Exon2R (5′-CCGTGGGTGGGAAAAGAG-3′), Exon2F (5′-CATTCTCTTTTCCCAC-3′), Exon3R (5′-TTTCCGTGGGTGTTTCAC-3′), Exon4F (5′-CCTCTACCCTGCCAC CAC -3′), Exon5R (5′-ACCATCACGCTCTCTCCC-3′). All introns except intron#4, which was sequenced completely, were sized by estimation of PCR product sizes using the following combinations of primers: Exon1F & Exon2R; Exon2F & Exon3R; Exon 4F & Exon5R. PCR reactions were performed using standard conditions with 30 cycles of the following parameters: 94°, 1 min; 55°, 1 min; 72°, 2 min. PCR products were separated on a 0.8% agarose gel and sizes were estimated based on comparison with lambda/Hind III and lambda/BstI molecular weight standards (Promega). The 5′ non-coding sequence was obtained using an exon1 reverse primer (5′-GTCCCCGAGAAGCAAACC-3′), the Exon2R primer, above, and an upstream forward primer (5′-CCCACGTCACAAGTCTGG-3′) designed to the sequenced upstream region. The reported putative promoter sequence was confirmed by at least three separate sequencing reactions.

### Results

A genomic library made from the 129/SvJ mouse strain was screened using a probe made from the coding region of the mouse Aop2 cDNA. Twenty one individual positive clones were identified, and Southern blotting was used to distinguish unique sequences. The results from this analysis suggested at least three distinct genes; these included Aop2 and two highly related intronless genes.

One group of phage clones contained overlapping sequences of the true Aop2 gene, as demonstrated by over 99% nucleotide identity to the Aop2 cDNA isolated from the C57BL/6J and DBA/2J strains of mice. The original publication of the Aop2 cDNA reported two nucleotides in the coding region that were divergent between the DBA/2J and C57BL/6J strains; one of which is at cDNA position#80, which does not change the coded amino acid, and the second is at nucleotide#439, which is adenine in DBA/2J and cytosine in C57BL/6J. The corresponding amino acids at this position are aspartic acid in DBA/2J and alanine in C57BL/6J. Like that of DBA/2J the Aop2 gene from the 129/SvJ strain encodes aspartic acid at nucleotide#439. The Aop2 allele from 129/SvJ differs from DBA/2J only at the variant nucleotide#80 which is guanine in 129/SvJ, encoding the same amino acid as all of the other strains analyzed.

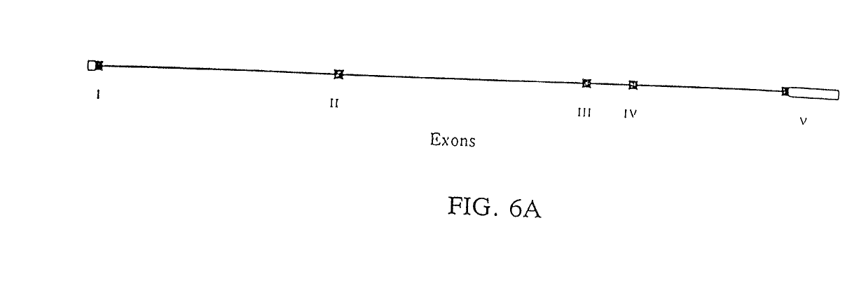

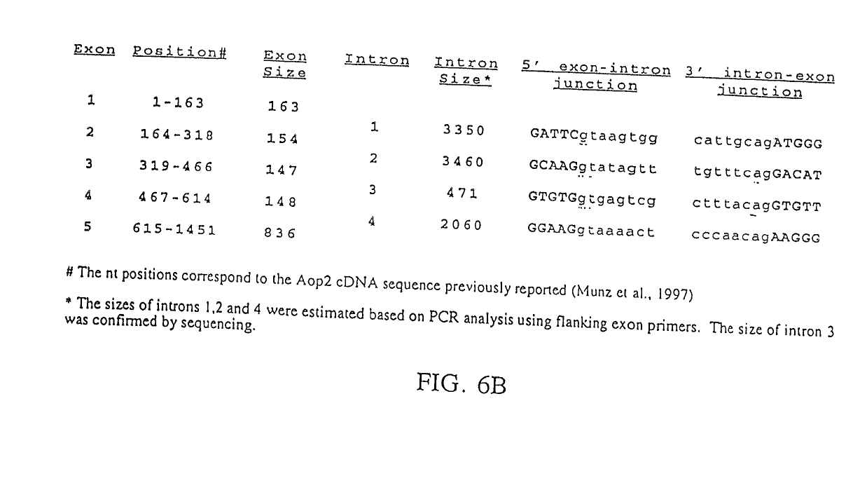

Further sequence analysis of these clones revealed that the gene consists of five exons and four introns, spanning approximately 10.7 kb. The genomic structure of the mouse Aop2 gene is shown in FIG. 6. The location of exons within the Aop2 gene are indicated in panel A. As shown, all four introns are contained within the coding region of the gene. The precise size and location of exons and introns are shown in panel B, as well as the confirmed sequence of the intron/exon boundaries. The nucleotide numbers correspond to the presumed full length Aop2 cDNA as previously reported (Munz et al., J. Biochem. 326: 579-585 (1997); Frank et al., Oncogene 14: 915-921 (1997)). While the intron sizes range from 471 bp to approximately 3.5 kb in length, the first four exons are relatively close in size, ranging between 147 and 163 bp. The 3′ end of the coding region and the 667 bp 3′ UTR are found within the last exon. The 3′ UTR of Aop2 from the 129/SvJ strain was also compared with a partial 3′ UTR we have isolated from the C57BL/6 strain, and the previously reported full length 3′UTR from BALB/cJ [Munz et al., J. Biochem. 326: 579-585 (1997), #2672]. We found a single nucleotide difference at nucleotide #1037, which is thymine in C57BL/6J and cytosine in 129/SvJ and BALB/cJ. These are the only strains for which the 3′ UTR of Aop2 is known.

In order to begin to understand how Aop2 may be regulated in vivo the region upstream of the 5′ most cDNA sequence, as previously reported (Munz et al., J. Biochem. 326: 579-585 (1997); Frank et al., Oncogene 14: 915-921 (1997)), was isolated and sequenced. Approximately 500 nucleotides of this upstream region are shown in FIG. 7. Analysis of this sequence identified no consensus TATA-box. However, several potential SP 1 binding sites are located within 60 nucleotides upstream of the presumed +1 transcription start site, suggesting that basal transcription of Aop2, like many other ubiquitously expressed genes, may be regulated by these transcription factors. There is also a sequence which resembles a transcriptiation initiation sequence commonly found in TATA-less promoters (CTCANTCT). The actual sequence differs from this consensus by a single nucleotide insertion. However, this element is located 20 nucleotides downstream of the presumed transcription start site, as previously reported.

Several additional consensus recognition sequences for known transcription factors are found in the putative proximal promoter. As shown in FIG. 7, these include potential binding sites for USF (upstream stimulatory factor) and SREBP (sterol response element binding protein), two DNA binding proteins that have been demonstrated to be important in the regulation of a number of genes involved in lipid metabolism. The putative USF recognition sequence matches 100% to the known binding site, while the putative SREBP binding site contains 11 out of 12 nucleotides of the consensus sequence. There is also a consensus recognition sequence for ADR1 (alcohol dehydrogenase regulated gene 1), which is identical to the known binding site. In addition, this region contains several potential binding sites for Heat Shock Factor (HSF), all of which match perfectly to the consensus recognition sequence.

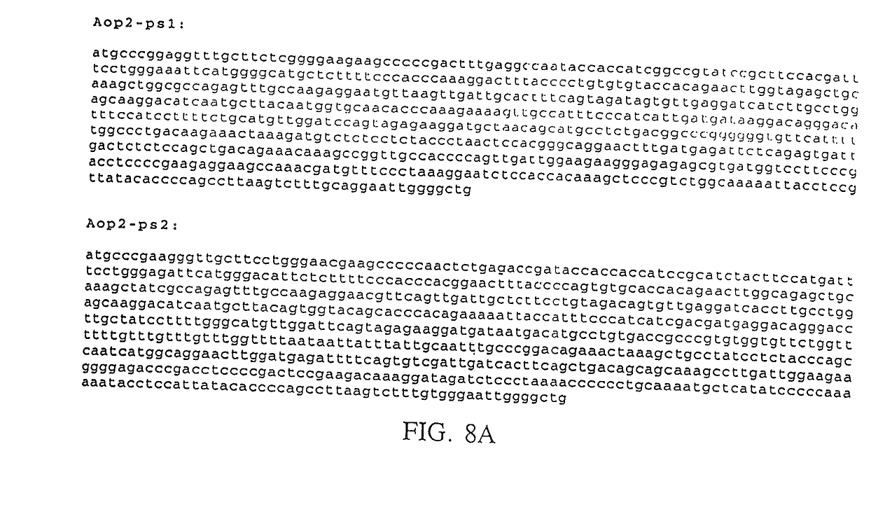

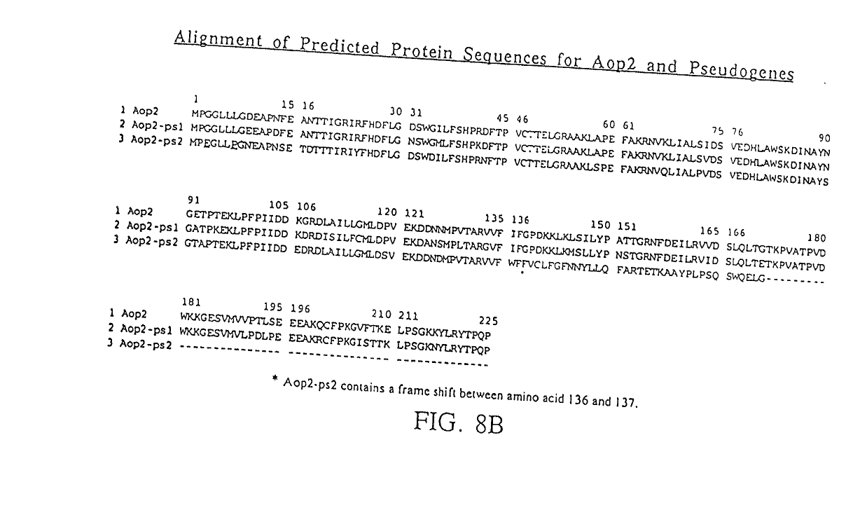

In addition to the Aop2 gene, two highly related genes were also identified from the original screen. These genes have been named Aop2-rs1 and Aop2-rs2 (Aop2-related sequence 1 and 2). FIG. 8A displays a comparison of the nucleotide sequences of all three genes, corresponding to the coding region of Aop2. As shown, Aop2-rs1 shares 93% nucleotide identity with Aop2 in the coding region. Each nucleotide difference in Aop2-rs1 is the result of a base substitution, resulting in several amino acid changes but an intact open reading frame. In contrast, Aop2-rs2 appears much more divergent, sharing only 80% nucleotide identity with Aop2, including 67 nucleotide substitutions, 24 nucleotide deletions and 39 nucleotide insertions. The first nucleotide deletion interrupts codon# 114 and disrupts the reading frame, resulting in a truncated protein, which is 119 amino acids in length. An alignment of the predicted protein products with the known Aop2 protein sequence is shown in FIG. 8B. While the presumed Aop2-rs1 protein shows 86% identity to Aop2, Aop2-rs2 shares only 42% amino acid identity.

### X. REFERENCES

The following references, to the extent that they provide exemplary procedural or other details supplementary to those set forth herein, are specifically incorporated herein by reference:

Albarosa et al., Deletion mapping of gliomas suggest the presence of two small regions for candidate tumor-suppressor genes in a 17-cM interval on chromosome 10 q. Am J. Genet., 58:1260-1267, 1996.

Baichwal and Sugden, Vectors for gene transfer derived from animal DNA viruses: Transient and stable expression of transferred genes. In: Gene Transfer, Kucherlapati R, ed., New York, Plenum Press, pp. 117-148, 1986.

Banka, C. L., High density lipoprotein and lipoprotein oxidation. Current Opinions Lipidol. 7:139-142, 1996.

Barany and Merrifield, The Peptides, Gross and Meienhofer, eds., Academic Press, New York, pp. 1-284, 1979.

Beier D R “Single-strand conformation polymorphism (SSCP) analysis as a tool for genetic mapping.” Mamm. Genome, 4 (11) p627-31, 1993.

Beier D R; Dushkin H; Sussman DJ “Mapping genes in the mouse using single-strand conformation polymorphism analysis of recombinant inbred strains and interspecific crosses.” Proc Natl Acad Sci USA, 89 (19) p9102-6 1992.

Bellus, J. Macromol. Sci. Pure Appl. Chem, A31(1): 1355-1376, 1994.

Benvenisty and Neshif, Direction introduction of genes into rats and expression of the genes. Proc. Nat. Acad. Sci. USA, 83:9551-9555, 1986.

Bianchi et al., Mutations in transcript isoforms of the neurofibromatosis 2 gene in multiple human tumor types. Nature Genetics, 6:185-192, 1994.

Bishop, J. M., Molecular themes in oncogenesis. Cell, 64:2351-248, 1991.

Boring et al., Cancer Statistics, 1994 CA, 43:7-26, 1994.

Bowry et al., High density lipoprotein is the major carrier of lipid hydroperoxides in human blood plasma from fasting donors. Proc. Nat'l Acad. Sci. USA 89:10316-10320, 1992.

Brinster et al., Proc. Nat'l Acad. Sci. USA, 82: 4438-4442, 1985.

Campbell, in Monoclonal Antibody Technology, Laboratory Techniques in Biochemistry and Molecular Biology Vol. 13, Burden and Von Knippenberg, Eds. pp. 75-83, Amsterdam, Elseview, 1984

Capaldi et al., Biochem. Biophys. Res. Comm., 76:425, 1977

Cech et al., “In vitro splicing of the ribosomal RNA precursor of Tetrahymena: involvement of a guanosine nucleotide in the excision of the intervening sequence,“Cell, 27:487-496, 1981.

Chae et al., Cloning and sequencing of thiol-specific antioxidant from mammalian brain. Proc. Nat'l Acad. Sci. USA 91:7017-7021, 1994.

Chang et al., Foreign gene delivery and expression in hepatocytes using a hepatitis B virus vector. Hepatology, 14:124A, 1991.

Chen and Okayama, High-efficiency transfection of mammalian cells by plasmid DNA. Mol. Cell Biol., 7:2745-2752, 1987.

Coffin, Retroviridae and Their Replication. In: Virology, Fields et al., eds., Raven Press, New York, pp. 1437-1500,1990.

Collet et al., Protein kinase activity associated with the avian sarcoma virus src gene product. Proc. Natl. Acad. Sci. USA, 75:2021-2024, 1978.

Couch et al., “Immunization with types 4 and 7 adenovirus by selective infection of the intestinal tract,” Am. Rev. Resp. Dis., 88:394-403, 1963.

Coupar et al., A general method for the construction of recombinant vaccinia virus expressing multiple foreign genes, Gene, 68:1-10, 1988.

Culver et al., In vivo gene transfer with retroviral vector-producer cells for treatment of experimental brain tumors. Science, 256:1550-1552, 1992.

Dubensky et al., Direct transfection of viral and plasmid DNA into the liver or spleen of mice. Proc. Nat. Acad. Sci. USA, 81:7529-7533, 1984.

Elliott et al., Mapping the gene for LTW-4, a 26,000 molecular weight major protein of mouse liver and kidney. Mol. Gen. Genet. 180:17-22.

Elliott, R., Mouse variants studied by two-dimensional electrophoresis. Mouse News Lett. 61:59, 1979.

Elliott, R., Use of two-dimensional electrophoresis to identify and map new mouse genes. Genetics 91:295-308, 1979.

EP 329 822, Davey et al.

Fearron and Vogelstein, A genetic model for colorectal tumorigenesis. Cell, 61:759-767, 1990.

Fechheimer et al., Transfection of mammalian cells with plasmid DNA by scrape loading and sonication loading. Proc. Natl. Acad. Sci. USA, 84:8463-8467, 1987.

Ferkol et al, Regulation of the phosphoenolpyruvate carboxykinase/human factor IX gene introduced into the livers of adult rats by receptor-mediated gene transfer. FASEB J., 10 7:1081-1091, 1993.

Fodor et al., Light-directed, spatially addressable parallel chemical synthesis. Science, 251:767-773, 1991.

Forster and Symons, “Self-cleavage of plus and minus RNAs of a virusoid and a structural model for the active sites,” Cell, 49:211-220, 1987

Foulds, The natural history of cancer. J. Chronic Dis., 8:2-37, 1958.

Fraley et al., Entrapment of a bacterial plasmid in phospholipid vesicles: Potential for gene transfer. Proc. Natl. Acad. Sci. USA, 76:3348-3352, 1979.

Freifelder, Physical Biochemistry Applications to Biochemistry and Molecular Biology, 2nd ed. Wm. Freeman and Co., New York, N.Y., 1982.

Freshner, Animal Cell Culture: A Practical Approach, 2nd ed., Oxford/New York, IRL Press, Oxford University Press, 1992.

Friedmann, Progress toward human gene therapy. Science, 244:1275-1281, 1989.

Frohman, In: PCR Protocols: A Guide To Methods And Applications, Academic Press, N.Y., 1990.

GB 2 202 328

Gefter et al., Somatic Cell Genet., 3: 231-236, 1977.

Gerlach et al., “Construction of a plant disease resistance gene from the satellite RNA of tobacco rinspot virus,” Nature (London), 328:802-805, 1987.

Ghosh-Choudhury et al., EMBO J., 6:1733-1739, 1987.

Ghosh and Bachhawat, Targeting of Liposomes to Hepatocytes. In: Liver Diseases, Targeted Diagnosis and Therapy Using Specific Receptors and Ligands. Wu et al., eds., Marcel Dekker, New York, pp. 87-104, 1991.

Goding, 1986, In: Monoclonal Antibodies: Principles and Practice, 2d ed., Academic Press, Orlando, Fla., pp. 60-61, and 71-74, 1986.

Goldman D ; Pikus HJ “Fourteen genetically variant proteins of mouse brain: discovery of two new variants and chromosomal mapping of four loci.” Biochem, 24 (3-4) p183-94, 1986.

Gomez-Foix et al., J. Biol. Chem., 267:25129-25134, 1992.

Gopal, Gene transfer method for transient gene expression, stable transfection, and cotransfection of suspension cell cultures. Mol. Cell Biol., 5:1188-1190, 1985.

Graham & Prevec, Biotechnology, 20:363-390, 1992.

Graham and Prevec, In: E. J. Murray (ed.), Methods in Molecular Biology: Gene Transfer and Expression Protocol, Clifton, N.J.: Humana Press, 7:109-128, 1991.

Graham and Prevec, In: Methods in Molecular Biology: Gene Transfer and Expression Protocol, E. J. Murray, ed., Humana Press, Clifton, N.J., 7:109-128, 1991.

Graham and van der Eb, A new technique for the assay of infectivity of human adenovirus 5 DNA. Virology, 52:456-467, 1973.

Graham et al., Characteristics of a human cell line transformed by DNA from human adenovirus type 5.J. Gen. Virol., 36:59-72, 1977.

Grunhaus and Horwitz, Adenovirus as cloning vector. Seminar in Virology, 3:237-252, 1992.

Guertin et al, Malondialdehyde-modified high-density lipoprotein cholesterol: plasma removal, tissue distribution and biliary sterol secretion in rats. Biochim. Biophys. Acta 1214:137-142.

Gyapay et al., The 1993-94 Genethon human genetic linkage map. Nat. Genet., 7:246-339, 1994.

Hacia et al., Detection of heterozygous mutations in BRCA1 using high density oligonucleotide arrays and two-color fluorescence analysis. Nature Genetics, 14:441-447, 1996.

Hahn & Subbiah, Significant association of lipid peroxidation products with high density lipoprotein. Biochem. Mol. Biol. Int. 33:699-704, 1994.

Harland and Weintraub, Translation of mammalian mRNA injected into Xenopus oocytes is specifically inhibited by antisense RNA. J. Cell Biol., 101:1094-1099, 1985.

Harlow, E. & Lane, D. (1988) Antibodies: A Laboratory Manual (Cold Spring Harbor Laboratory, Cold Spring Harbor, N.Y.).

Henson et al., The retinoblastoma gene is involved in malignant progression of astrocytomas. Ann. Neurol., 36:714-721, 1994.

Hermonat and Muzycska, Use of adenoassociated virus as a mammalian DNA cloning vector: Transduction of neomycin resistance into mammalian tissue culture cells. Proc. Nat. Acad. Sci. USA, 81:6466-6470, 1984.

Hersdorffer et al., DNA Cell Biol., 9:713-723, 1990.

Herz and Gerard, Proc. Nat'l Acad. Sci. USA, 90:2812-2816, 1993.

Horwich, et al., Synthesis of hepadnavirus particles that contain replication-defective duck hepatitis B virus genomes in cultured HuH7 cells. J. Virol., 64:642-650, 1990.

Howell and Lane, In: Antibodies: A Laboratory Manual, Cold Springs Harbor Laboratory, 1988.

Hunter, T., Cooperation between oncogenes. Cell, 64-249-270, 1991.

Iakoubova et al., LTW4 protein on mouse chromosome 1 is a member of a family of antioxidant proteins. Genomics (in press) 1997.

Innis et al., PCR Protocols, Academic Press, Inc., San Diego Calif., 1990.

Johnson et al., Peptide Turn Mimetics” IN: Biotechnology And Pharmacy, Pezzuto et al., eds., Chapman and Hall, New York, 1993.

Jones and Shenk, Cell, 13:181-188, 1978.

Joyce, “RNA evolution and the origins of life,” Nature, 338:217-244, 1989.

Kaneda et al., Increased expression of DNA cointroduced with nuclear protein in adult rat liver. Science, 243:375-378, 1989.

Karlsson et al., EMBO J, 5:2377-2385, 1986.

Kato et al., Expression of hepatitis B virus surface antigen in adult rat liver. J. Biol. Chem., 266:3361-3364, 1991.

Kim and Cech, “Three dimensional model of the active site of the self-splicing rRNA precursor or Tetrahymena,” Proc. Natl. Acad. Sci. USA, 84:8788-8792, 1987.

Klein et al., High-velocity microprojectiles for delivering nucleic acids into living cells. Nature, 327:70-73, 1987.

Klimov A N; Gurevich V S; Nikiforova A A; Shatilina L V; Kuzmin A A; Plavinsky S L; Teryukova N P “Antioxidative activity of high density lipoproteins in vivo.” Atherosclerosis, 100 (1) p13-8, 1993.

Kohler and Milstein, Eur. J Immunol., 6:511-519, 1976.

Kohler and Milstein, Nature, 256:495-497, 1975.

Kok et al., A homozygous deletion in a small cell lung cancer cell line involving a 3p21 region with a marked instability in yeast artificial chromosomes. Cancer Res. 54:4183-4187, 1994.

Kwoh et al., Proc. Nat. Acad. Sci. USA, 86: 1173, 1989.

Kyte and Doolittle, J. Mol. Biol., 157(1):105-132, 1982.

Le Gal La Salle et al., Science, 259:988-990, 1993.

Levrero et al., Gene, 101:195-202, 1991.

Macejak and Sarnow, Nature, 353:90-94, 1991.

Manipulating the Mouse Embryo: A Laboratory Manual, 2nd ed., Hogan et al., eds., Cold Spring Harbor Laboratory Press, 1994.

Mann et al., Construction of a retrovirus packaging mutant and its use to produce helper-free defective retrovirus. Cell, 33:153-159, 1983.

Marcel et al., Lipid peroxidation changes the expression of specific epitopes of apolipoprotein A-1.J Biol. Chem. 264:19942-19950, 1989.

Markowitz et al., J. Virol., 62:1120-1124, 1988.

Merrifield, Science, 232: 341-347, 1986

Michel and Westhof, “Modeling of the three-dimensional architecture of group I catalytic introns based on comparative sequence analysis,” J. Mol. Biol., 216:585-610, 1990.

Mulligan, Science, 260:926-932, 1993.

Murakami et al., Suppression of the malignant phenotype of human prostate cancer cell line PPC-1 by introduction of normal fragments of human chromosome 10. Cancer Res., 56:2157-2160, 1996.

Myers, EP 0273085

Netto et al., J. Biol. Chem. 271 (26):15315-21, 1996.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, Liposome-mediated DNA transfer in eukaryotic cells. Biochim. Biophys. Acta, 721:185-190, 1982.

Nicolau et al., Methods Enzymol., 149:157-176, 1987.

Ohara et al., Proc. Nat'l Acad. Sci. USA, 86:5673-5677, 1989

Ohkawa et al., Assay for lipid peroxides in animal tissues by thiobarbituric acid reaction. Anal. Biochem. 95:351-358, 1979.

Paigen B; Havens M B; Morrow A “Effect of 3-methylcholanthrene on the development of aortic lesions in mice.” Cancer Res. 45 (8) p3850-5, 1985

Paigen B; Morrow A; Brandon C; Mitchell D; Holmes P “Variation in susceptibility to atherosclerosis among inbred strains of mice.” Atherosclerosis, 57 (1) p65-73, 1985

Paigen et al., Ath-1, a gene determining atherosclerosis susceptibility and high density lipoprotein levels in mice. Proc. Nat'l Acad. Sci. USA 84:3763-3767, 1987a.

Paigen et al., Atherosclerosis susceptibility differences among progenitors of recombination inbred strains of mice. Arteriosclerosis 10:316-323, 1990

Paigen et al., Genetic analysis of murine strains C57BL/6J and C3H/HeJ to confirm the map position of Ath-1, a gene determining atherosclerosis susceptibility. Biochem. Genetics 25:501-511, 1987b.

Paigen et al., Genetic analysis of strains C57BL/6J and BALB/cJ for Ath-1, a gene determining atherosclerosis susceptibility in mice. Biochem. Genetics 25:881-892, 1987c.

Parthasarathy et al., High-density lipoprotein inhibits the oxidative modification of low-density lipoprotein. Biochim. Biophys. Acta 1044:

Paskind et al, Dependence of moloney murine leukemia virus production on cell growth. Virology, 67:242-248, 1975.

PCT/US87/00880

PCT/US89/01025

Pease et al., Light-generated oligonucleotide arrays for rapid DNA sequence analysis. Proc. Natl. Acad. Sci. USA, 91:5022-5026, 1994.

Pelletier and Sonenberg, Nature, 334:320-325, 1988.

Perales et al., Gene transfer in vivo: Sustained expression and regulation of genes introduced into the liver by receptor-targeted uptake. Proc. Natl. Acad. Sci. 91:4086-4090, 1994.

Pignon et al., Hum. Mutat., 3: 126-132, 1994.

Potteret al., Proc. Nat'l Acad. Sci. USA, 81:7161-7165, 1984.

Racher et al., Biotechnology Techniques, 9:169-174, 1995.

Racine R R; Langley C H “Genetic analysis of protein variations in Mus musculus using two-dimensional electrophoresis.” Biochem Genet, 18 (1-2) p185-97, 1980

Ragot et al., Nature, 361:647-650, 1993.

Rasheed et al., Oncogene, 11:2243-2246, 1995.

Reinhold-Hurek and Shub, “Self-splicing introns in the tRNA genes of widely divergent bacteria,” Nature, 357:173-176, 1992.

Remington's Pharmaceutical Sciences, 15th ed., pp. 1035-1038 and 1570-1580.

Rich et al., Hum. Gene Ther., 4:461-476, 1993.

Ridgeway, Mammalian Expression Vectors, In: Vectors: A Survey of Molecular Cloning Vectors and Their Uses, Rodriguez et al., eds., Stoneham: Butterworth, pp. 467-492, 1988.

Rippe et al., Mol. Cell Biol., 10:689-695, 1990.

Rosenfeld et al., In vivo transfer of the human cystic fibrosis transmembrane conductance regulator gene to the airway epithelium. Cell, 68:143-155, 1992.

Rosenfeld et al., Science, 252:431-434, 1991.

Roux et al., A versatile and potentially general approach to the targeting of specific cell types by retroviruses: Application to the infection of human cells by means of major histocompatibility complex class I and class II antigens by mouse ecotropic murine leukemia virus-derived viruses. Proc. Nat ′ Acad. Sci. USA, 86:9079-9083, 1989.

Russell and Rubinstein, Tumors of the neuroepithelial origin, In: Pathology of Tumors of the Nervous System, 5th ed., Williams and Wilkins, eds., pp. 82-219, 1989.

Sambrook et al., In: Molecular Cloning: A Laboratory Manual, 2d Ed., Cold Spring Harbor Laboratory Press, Cold Spring Harbor, N.Y., 1989.

Sarver et al., “Ribozymes as potential anti-HIV-1 therapeutic agents,” Science, 247:1222-1225, 1990.

Sarver, et al, Science, 247:1222-1225, 1990.

Scanlon et al., “Ribozyme-mediated cleavages of c-fos mRNA reduce gene expression of DNA synthesis enzymes and metallothionein,” Proc. Nat'l. Acad. Sci. USA, 88:10591-10595, 1991.

Senault et al., Effect of apoprotein cross-linking on the metabolism of human HDL₃ in rat. Biochim. Biophys. Acta 1046:81-88, 1990.

Shoemaker et al., Quantitative phenotypic analysis of yeast deletion mutants using a highly parallel molecular bar-coding strategy. Nature Genetics 14:450-456, 1996.

Sonoda et al., Cancer Res., 55:2166-2168, 1995.

Stewart and Young, Solid Phase Peptide Synthesis, 2d. ed., Pierce Chemical Co., 1984.

Stratford-Perricaudet and Perricaudet, Gene transfer into animals: the promise of adenovirus. In: Human Gene Transfer, O. Cohen-Haguenauer et al., eds., John Libbey Eurotext, France, pp. 51-61, 1991.

Stratford-Petticaudet et al., Evaluation of the transfer and expression in mice of an enzyme-encoding gene using a human adenovirus vector. Hum. Gene. Ther., 1:241-256, 1990.

Tam et al., J. Am. Chem. Soc., 105:6442, 1983.

Temin, Retrovirus vectors for gene transfer: Efficient integration into and expression of exogenous DNA in vertebrate cell genome. In: Gene Transfer, Kucherlapati R, ed., New York, Plenum Press, pp. 149-188, 1986.

Top et al., “Immunization with live types 7 and 4 adenovirus vaccines. II. Antibody response and protective effect against acute respiratory disease due to adenovirus type 7,” J. Infect. Dis., 124:155-160, 1971.

Tsuji et al., Mammalim antioxidant protein complements alkylhydroperoxide reductase (ahpC) mutation in Escherichia coli. Biochem. J 307:377-381, 1995.

Tur-Kaspa et al., Use of electroporation to introduce biologically active foreign genes into primary rat hepatocytes. Mol. Cell Biol., 6:716-718, 1986.

U.S. Pat. No. 4,873,191, Wagner and Hoppe

U.S. Pat. No. 5,279,721

Varmus et al., Cell 25:23-36, 1981.

Voullaire et al., A functional marker centromere with no detectable alpha-satellite, satellite III, or CENP-B protein: activation of a latent centromere? Am. J. Hum. Genet., 52:1153-1163.

Wagner et al., Science, 260:1510-1513, 1993.

Walker et al., Proc. Nat'l Acad. Sci. USA, 89:392-396 1992.

Weinberg, Positive and negative controls on cell growth. Biochemistry, 28:8263-8269, 1989.

Wilson D L; Hall M E; Stone G C; Rubin R W “Some improvements in two-dimensional gel electrophoresis of proteins. Protein mapping of eukaryotic tissue extracts.” Anal Biochem, 83 (1) p33-44, 1977.

WO 88/10351, Gingeras et al.

WO 89/06700, Miller et al.

WO 90/07641, filed Dec. 21, 1990.

Wong et al., Appearance of b-lactamase activity in animal cells upon liposome mediated gene transfer, Gene, 10:87-94, 1980.

Wu and Wu, Adv. Drug Delivery Rev., 12:159-167, 1993.

Wu and Wu, Evidence for targeted gene delivery to HepG2 hepatoma cells in vitro. Biochemistry, 27:887-892, 1988.

Wu and Wu, Receptor-mediated in vitro gene transfections by a soluble DNA carrier system. J. Biol. Chem., 262:4429-4432, 1987.

Wu et al., Genomics, 4:560, 1989.

Yamamoto et al., Cloning of a housekeeping-type gene (MER5) preferentially expressed in murine erythroleukemia cells. Gene 80:337-343, 1989.

Yang et al., In vivo and in vitro gene transfer to mammalian somatic cells by particle bombardment. Proc. Natl. Acad. Sci. USA, 87:9568-9572, 1990.

Zelenin et al., High-velocity mechanical DNA transfer of the chloramphenicol acetyltransferase gene into rodent liver, kidney and mammary gland cells in organ explants and in vivo. FEBS Lett., 280:94-96, 1991.

What is claimed is:

1. An isolated polypeptide designated AOP2.

2. The isolated polypeptide of claim 1, wherein the polypeptide is a human polypeptide.

3. The isolated polypeptide of claim 1, wherein the polypeptide is a murine polypeptide.

4. The isolated polypeptide of claim 2, wherein the polypeptide has the sequence set forth in SEQ ID NO:2.

5. The isolated polypeptide of claim 3, wherein the polypeptide has the sequence set forth in SEQ ID NO:4.

6. An antigen composition comprising an Aop2 polypeptide or a fragment thereof and a pharmaceutically acceptable buffer or diluent.

7. The antigen composition of claim 6, wherein the polypeptide is a human polypeptide.

8. The antigen composition of claim 6, wherein the polypeptide is a murine polypeptide.

9. The antigen composition of claim 7, wherein the polypeptide has the sequence set forth in SEQ ID NO:2.

10. The antigen composition of claim 8, wherein the polypeptide has the sequence set forth in SEQ ID NO:4.

11. A nucleic acid encoding an AOP2 polypeptide.

12. The nucleic acid of claim 11, wherein the polypeptide is a human polypeptide.

13. The nucleic acid of claim 11, wherein the polypeptide is a murine polypeptide.

14. The nucleic acid of claim 12, wherein the polypeptide has the sequence set forth in SEQ ID NO:2.

15. The nucleic acid of claim 13, wherein the polypeptide has the sequence set forth in SEQ ID NO:4.

16. A oligonucleotide comprising at least about 10 consecutive bases of the nucleic acid sequence set forth in SEQ ID NO:1 or SEQ ID NO:3.

17. The oligonucleotide of claim 16, wherein said oligonucleotide is at least about 15 consecutive bases of the nucleic acid set forth in SEQ ID NO:1 or SEQ ID NO:3.

18. The oligonucleotide of claim 17, wherein said oligonucleotide is at least about 20 consecutive bases of the nucleic acid set forth in SEQ ID NO: 1 or SEQ ID NO:3.

19. The oligonucleotide of claim 18, wherein said oligonucleotide is at least about 25 consecutive bases of the nucleic acid set forth in SEQ ID NO: 1 or SEQ ID NO:3.

20. The oligonucleotide of claim 19, wherein said oligonucleotide is at least about 30 consecutive bases of the nucleic acid set forth in SEQ ID NO: 1 or SEQ ID NO: 3.

21. The oligonucleotide of claim 20, wherein said oligonucleotide is at least about 35 consecutive bases of the nucleic acid set forth in SEQ ID NO: 1 or SEQ ID NO:3.

22. The oligonucleotide of claim 21, wherein said oligonucleotide is at least about 40 consecutive bases of the nucleic acid set forth in SEQ ID NO: 1 or SEQ ID NO:3.

23. The oligonucleotide of claim 22, wherein said oligonucleotide is at least about 45 consecutive bases of the nucleic acid set forth in SEQ ID NO:1 or SEQ ID NO:3.

24. The oligonucleotide of claim 16, wherein said oligonucleotide is at least about 50 consecutive bases of the nucleic acid set forth in SEQ ID NO: 1 or SEQ ID NO:3.

25. A method for diagnosing a predisposition to atherosclerotic lesions in a subject comprising:
(i) obtaining a sample from said subject; and
(ii) evaluating said sample for the presence of an AOP2 polypeptide.

26. The method of claim 25, wherein said sample is selected from the group consisting of heart, artery, vein, skin, muscle, facia, brain, prostate, breast, endometrium, lung, pancreas, small intestine, blood cells, liver, testes, ovaries, colon, skin, stomach, esophagus, spleen, lymph node, bone marrow or kidney, lymph fluid, ascites, serous fluid, pleural effusion, sputum, cerebrospinal fluid, lacrimal fluid, stool and urine.

27. The method of claim 25, wherein said subject is a human.

28. The method of claim 25, wherein said evaluating comprises determining the antioxidant activity of an AOP2 polypeptide of said sample.

29. The method of claim 25, wherein said evaluating comprises determining the level of an AOP2 polypeptide in cells of said sample.

30. The method of claim 29, wherein said determining comprises quantitative PCR.

31. The method of claim 29, wherein said determining comprises contacting said sample with an antibody that binds immunologically to an AOP2 polypeptide.

32. The method of claim 25, wherein said evaluating comprises determining the sequence of a nucleic acid from said sample that encodes an AOP2 polypeptide.

33. A method for screening a compound for AOP2 stimulatory activity comprising:
(i) providing an AOP2 polypeptide having antioxidant activity;
(ii) contacting said AOP2 polypeptide with a candidate stimulator; and
(iii) determining the antioxidant activity of said AOP2 polypeptide in the presence and absence of said candidate stimulator.

34. A method for screening a compound for antioxidant stimulatory activity comprising:
(i) providing a cell comprising an nucleic acid encoding an active AOP2 polypeptide;
(ii) contacting said cell with a candidate stimulator; and
(iii) determining the antioxidant activity in said cell in the presence and absence of said candidate stimulator.

35. The method of claim 34, wherein said cell is located in a non-human animal.

36. A method for screening a compound for anti-atherosclerotic activity comprising:
(i) providing a lipid;
(ii) contacting said lipid with a candidate antioxidant; and
(iii) determining the oxidation state of said lipid.

37. A monoclonal antibody that binds immunologically to an AOP2 polypeptide.

38. A polyclonal antisera, antibodies of which bind immunologically to an AOP2 polypeptide.

39. An expression vector comprising a nucleic acid encoding an AOP2 polypeptide, said nucleic acid positioned in operable relation to a promoter.

40. A recombinant host cell comprising a nucleic acid encoding an AOP2 polypeptide, said nucleic acid positioned in operable relation to a promoter.

41. A method for increasing AOP2 function in a cell comprising:
(i) providing a nucleic acid encoding an AOP2 polypeptide having antioxidant activity, said nucleic acid positioned in operable relation to a promoter; and
(ii) contacting said nucleic acid with said cell under conditions permitting the uptake of said nucleic acid.

42. The method of claim 41, wherein said AOP2 polypeptide is a human polypeptide.

43. The method of claim 42, wherein said AOP2 polypeptide has the sequence set forth in SEQ ID NO:2.

44. The method of claim 41, wherein said nucleic acid further comprises an expression vector.

45. The method of claim 44, wherein said expression vector is encapsulated in a liposome.

46. The method of claim 44, wherein said expression vector is a viral vector.

47. The method of claim 46, wherein said viral vector is selected from the group consisting of an adenoviral vector, a retroviral vector, a vaccinia viral vector, an adeno-associated viral vector or a herpesviral vector.

48. The method of claim 41, wherein said cell is located in a human subject.

49. The method of claim 41, wherein said cell is located in an experimental animal.

50. The method of claim 49, wherein said nucleic acid is administered intravenously.

51. The method of claim 41, wherein said promoter is selected from the group consisting of CMV, RSV and E1A.

52. A method of reducing atherosclerotic lesions in a subject comprising administering to said subject a lipid antioxidant composition.
